(19) United States
(12) Patent Application Publication
(10) Pub. No.: US 2024/0269352 A1
(43) Pub. Date: Aug. 15, 2024

(54) MINIATURIZED HYDROGEL AND USES THEREOF

(71) Applicant: UNIVERSITY OF MASSACHUSETTS, Boston, MA (US)

(72) Inventors: Siyuan Rao, Sunderland, MA (US); Sizhe Huang, Amherst, MA (US); Qianbin Wang, Sunderland, MA (US); Kayla Felix, Taunton, MA (US); Collin Maley, Medway, MA (US)

(21) Appl. No.: 18/440,095

(22) Filed: Feb. 13, 2024

Related U.S. Application Data
(60) Provisional application No. 63/445,346, filed on Feb. 14, 2023.

(51) Int. Cl. A61L 31/04

(52) U.S. Cl. CPC A61L 31/048

(57) ABSTRACT

A miniaturized hydrogel includes a reaction product of a hydroxyl-containing polymer, a primary crosslinker, and a secondary crosslinker, and water. The primary crosslinker and the secondary crosslinker are capable of reacting with the hydroxyl-containing polymer. The hydrogel is made by a method including contacting the hydroxyl-containing polymer, the primary crosslinker, and the secondary crosslinker under conditions effective to provide a crosslinked hydrogel; acidifying the crosslinked hydrogel; drying the crosslinked hydrogel under tension; and rehydrating the dried hydrogel to provide the miniaturized hydrogel. The miniaturized hydrogel can be particularly useful in various implantable medical devices.

### CROSS-REFERENCE TO RELATED APPLICATION

This application claims priority to U.S. Provisional Patent Application No. 63/445,346, filed on Feb. 14, 2023, the contents of which are hereby incorporated by reference herein in their entirety.

FEDERAL RESEARCH STATEMENT This invention was made with government support under award number 5R00MH120279 awarded by the National Institutes of Health. The government has certain rights in the invention.

### BACKGROUND

Bioelectronics made of viscoelastic materials exhibit motion-adaptive properties for brain-machine interfaces investigating complex neural circuits. To access delicate nerve structures, two-dimensional (2D) patterning microfabrication strategies for viscoelastic materials have yielded the advent of miniaturizing congruent interfaces. However, creating 3D architecture of viscoelastic bioelectronics for expansive implementation requires more accessible and scalable manufacturing approaches.

Accordingly, there remains a continuing need in the art for improved materials and methods that can be used to produce particular viscoelastic materials, especially for bioelectronics applications.

### SUMMARY

A miniaturized hydrogel comprises a reaction product of a hydroxyl-containing polymer, a primary crosslinker, and a secondary crosslinker; and water; wherein each of the primary crosslinker and the secondary crosslinker are capable of reacting with the hydroxyl-containing polymer; and wherein the hydrogel is made by a method comprising: contacting the hydroxyl-containing polymer, the primary crosslinker, and the secondary crosslinker under conditions effective to provide a crosslinked hydrogel; acidifying the crosslinked hydrogel; drying the crosslinked hydrogel under tension; and rehydrating the dried hydrogel to provide the miniaturized hydrogel.

A miniaturized hydrogel comprises 45 to 65, or 50 to 60, or 55 to 60 weight percent of a reaction product of a hydroxyl-containing polymer, a primary crosslinker, and a secondary crosslinker; and 35 to 55, or 40 to 50, or 40 to 45 weight percent water; wherein weight percent is based on the total weight of the miniaturized hydrogel; wherein the hydrogel comprises a plurality of nanocrystalline domains, wherein the nanocrystalline domains are present in an amount effective to provide the hydrogel with a total crystallinity of 5 to 20%; wherein the miniaturized hydrogel exhibits one or more of: a stretchability of greater than 100%, preferably 100 to 300%, more preferably 100 to 250%; or an elastic modulus of less than 35 MPa, or 20 to 32 MPa; or a refractive index of 1.35 to 1.45 at 480 nanometers; or a light transmission of greater than 95%.

In an aspect, a neural probe comprises the miniaturized hydrogel.

In an aspect, a microelectrode comprises the miniaturized hydrogel.

In an aspect, an implantable medical device comprises the miniaturized hydrogel.

A method for the manufacture of the miniaturized hydrogel comprises: contacting a hydroxyl-containing polymer, a primary crosslinker, and a secondary crosslinker under conditions effective to provide a crosslinked hydrogel; acidifying the crosslinked hydrogel; drying the crosslinked hydrogel under tension; and rehydrating the dried hydrogel to provide the miniaturized hydrogel.

The above described and other features are exemplified by the following figures and detailed description.

### BRIEF DESCRIPTION OF THE DRAWINGS

The following figures represent exemplary embodiments.

FIG. 1 is a schematic illustration of a hydrogel network of metamorphic polymers' amorphous-crystal transition (COMPACT): chemical cross-linking via glutaraldehyde (GA) and tetraethyl orthosilicate (TEOS), acidification with hydrochloric acid (HCl) to intervene polymer chain interaction, and static mechanical stretching under drying and annealing to re-orient nanocrystalline growth.

FIG. 2A shows images of TEOS-GA cross-linked hydrogel and GA cross-linked hydrogel at the pristine state.

FIG. 2B shows images of acidified TEOS-GA cross-linked hydrogel and GA cross-linked hydrogel at the desiccated state.

FIG. 2C shows images of acidified TEOS-GA cross-linked hydrogel and GA cross-linked hydrogel at the re-hydrated state.

FIG. 2D shows a plot of water content (%) of acidified TEOS-GA cross-linked hydrogel and GA cross-linked hydrogel at each state (pristine, desiccated, re-hydrated).

FIG. 3A shows shrinking behaviors of TEOS-GA cross-linked hydrogel membranes (4 wt. % TEOS and acidified). Top: a schematic of hydrogel membranes deposited on a substrate. Bottom: thickness of TEOS-GA cross-linked hydrogel membranes before and after shrinking. Data are mean±SD.

FIG. 3B shows shrinking acidified TEOS-GA cross-linked hydrogel fibers (4 wt. % TEOS, acidified, and 200% stretching). Top: a schematic of fibers in diameter and length. Bottom: shrinking percentage (%) in diameter versus TEOS content (wt. %) indicated by black dots, swelling percentage (%) in length versus TEOS content (wt. %) indicated by red dots.

FIG. 3C shows unconstrained shrinking of cross-linked hydrogel cylinders. Top: images of hydrogel cylinders with unconstrained: quarter coin, pristine cylinder, TEOS-GA cross-linked hydrogel cylinder (4 wt. % TEOS and acidified), GA cross-linked hydrogel cylinder from left to right. Bottom: shrinking percentage (%) in volume of TEOS-GA cross-linked hydrogel cylinders and GA cross-linked hydrogel cylinders. Data are mean±SD.

FIG. 4A shows (a) Fourier transform infrared (FTIR) spectroscopy of COMPACT (−) and COMPACT (+) hydrogels showing chemical bonds.

FIG. 4B shows differential scanning calorimetry (DSC) profiles of COMPACT (−) and COMPACT (+) indicating the crystallinity percentages.

FIG. 4C shows small-angle X-ray (SAXS) and wide-angle X-ray (WAXS) results of hydrogel materials in the dry state indicating that the size of nanocrystalline domains is around 3.5 nm, and an increasing nanocrystalline spacing with increased stretching percentages. Inset: SAXS and WAXS 2D patterns showing that the orientation of nanocrystals in the hydrogel before and after stretching.

FIG. 5A shows shrinking diagram of COMPACT (+) hydrogel fibers with shrinking percentage (%) versus stretching percentages (%) of chemically cross-linked PVA hydrogels with different TEOS content (wt. %) with acidification (shaded area) and without acidification.

FIG. 5B shows shrinking behaviors of COMPACT hydrogel fibers (4 wt. % TEOS) prepared in different sizes of molds. Each dot (mean±s.d.) represents one independent fiber (One-way ANOVA and Tukey's multiple comparisons test, F_{3,12}=0.9543, n.s.: not significant, p=0.4455).

FIG. 5C shows refractive index of COMPACT (+) hydrogel membranes versus TEOS content (wt. %) indicated by blue curve, normalized light transmittance of COMPACT (+) hydrogel membranes versus TEOS content (wt. %) indicated by red curve.

FIG. 6 shows stretchability of COMPACT (+) hydrogels with increased TEOS content (wt. %) and bending stiffness of COMPACT (+) hydrogels with increased TEOS content (wt. %).

FIG. 7 shows bending stiffness of COMPACT (3 wt. % TEOS) hydrogel fiber (n=4 independent samples) with identical cross-sections in comparison with silica fiber (diameter: 200 μm, n=3 independent samples).

FIG. 8A shows representative confocal images of astrocytes (GFAP, red), microglia/macrophages (Iba1, green), presence of immunoglobulin G (CD16/32, cyan), activated macrophages (CD68, yellow), and neuronal cells (NeuN, magenta) surrounding a 400 μm hydrogel fiber probe (n=3) and a 400 μm silica fiber (n=3) in mouse brain 14 days after implantation.

FIG. 8B shows measured total area of antibodies labeled cells of astrocytes (GFAP, red), microglia/macrophages (Iba1, green), macrophages (CD16/32, cyan), macrophages (CD68, yellow), and neuronal cells (NeuN, magenta) surrounding a 400 μm hydrogel fiber probe (n=3) and a 400 μm silica fiber in mouse brain 14 days after implantation. Each dot represents measurements from on confocal image around the implantation site, mean±sem.

FIG. 9A shows representative confocal images of astrocytes (GFAP, green) and microglia (Iba1, red) surrounding a 400 μm hydrogel fiber probe (n=6) in mouse brain 1 month after implantation.

FIG. 9B shows representative optical images of astrocytes (GFAP, green) and microglia/macrophages (Iba1, red) surrounding a 400 μm silica fiber probe (n=6) in mouse brain 1 month after implantation. Scale: 100 μm.

FIG. 9C shows counted GFAP fluorescence intensity from fibers implantation sites (left) and counted Iba1 fluorescence intensity from fibers implantation sites (right). (Unpaired student's t-test, GFAP: *p=0.0167; Iba1: *p=0.0108).

FIG. 9D shows counted total area of astrocytes around fibers implantation sites (left) and counted total area of microglia around fibers implantation sites (right). (Unpaired student's t-test, GFAP: *p=0.0251; Iba1: **p=0.0056). Each dot represents measurement from one confocal image around the implantation site, mean±sem.

FIG. 10 shows results of a stability assessment of diameter reduction of COMPACT hydrogel fibers (3 wt. % TEOS). Each dot (mean±s.d.) represents one independent fiber (two-way ANOVA and Tukey's multiple comparisons tests).

FIG. 11 shows results of a cytotoxicity assessment of COMPACT (+) hydrogels. Hydrogel fibers were incubated with Human Embryonic Kidney (HEK) 293 cell cultures. Calcein-AM (green) was used to stain living cells and ethidium homodimer-1 (red) was used to stain dead cells. Cell death rates are presented as mean±standard error (s.e.m., unpaired student's t-test).

FIG. 12A shows an illustration of light transmission in a core/cladding by total internal reflection.

FIG. 12B shows representative images of numerical apertures from COMPACT (−) core and COMPACT (+) core, respectively.

FIG. 12C shows schematics and optical images of COMPACT (+) core, COMPACT (+) core/plain cladding, COMPACT (+) core/GO cladding, respectively.

FIG. 12D shows light attenuation of the COMPACT fibers in a 1% agarose matrix indicates that the attenuation coefficient can be improved below 1 dB/cm.

FIG. 12E shows experimental scheme for the viral injection, optical fiber implantation, photometric recording and social behavior tests.

FIG. 12F shows representative images in mouse social interaction tests.

FIG. 12G shows a fiber photometry and setup with social behavioral assay,

FIG. 12H shows representative fluorescence intensity of normalized GCaMP6s (Z-score) in the VTA of freely exploring mice. Bars indicate social interaction time.

FIG. 13A shows a representative photograph of a carbon nanotubes (CNTs)-PVA hydrogel electrode as compared with a piece of human hair. Scale: 300 μm.

FIG. 13B shows a transmission electron microscopy (TEM) image of CNTs. Scale: 200 nm.

FIG. 13C shows impedance at 1 kHz (red dots) and diameters of the electrodes (blue dots) fabricated different stretching percentages (mean±s.d.). Each dot represents one independent hydrogel electrode.

FIG. 13D shows impedance at 1 kHz of electrodes fabricated with different CNTs concentrations (mean±s.d.). Each dot represents one independent hydrogel electrode.

FIG. 13E shows impedance at 1 kHz of electrodes (red dots) and diameters of the electrode (blue dots) fabricated with different sizes of molds (mean±s.d.). Each dot represents one independent hydrogel electrode.

FIG. 13F shows stability assessment on impedance (red dots) and diameters (blue dots) of hydrogel electrodes incubated in PBS at 37° C. (mean±s.d.). Each dot represents one independent hydrogel electrode.

FIG. 13G shows a schematic illustration of electrical recordings from mouse VTA with a CNTs-PVA electrode bundle (5 electrodes).

FIG. 13H shows representative electrophysiology recording signals from mouse VTA with CNTs-PVA hydrogel electrodes.

FIG. 13I shows a representative sorted spiking signal.

FIG. 13J shows a representative scanning electron microscopy (SEM) image at the cross-section of an integrated multifunctional neural probe containing a hydrogel optical core and two CNTs-PVA hydrogel electrodes. Scale: 100 μm.

FIG. 13K shows photographs of a hydrogel optoelectronic device (optrode) before implantation (left) and after implantation (right) in a Thy1::ChR2-EYFP mouse brain. Scale: 2 mm.

FIG. 14A shows confocal images of the expression of ChR2-EYFP in the VTA region of the mouse. Scale: 50 μm.

FIG. 14B shows representative in vivo electrical signals recorded with optrodes upon optical stimulation (blue bars, λ=473 nm, 0.5 Hz, pulse width 50 ms, 10 mW/mm²).

FIG. 14C shows amplitudes of electrical signals (n=3 mice) recorded with optical stimulation over 10 weeks post-implantation (λ=473 nm, 0.5 Hz, pulse width 50 ms, 10 mW/mm², mean±s.e.m.).

### DETAILED DESCRIPTION

Viscoelastic bioelectronics provide tissue-like interfaces for multifunctional modulation across single-cell and organ scales. In dynamic in vivo environments, such soft bio-interfaces can adapt to the persistent mechanical deformations of the living tissues, and consequently extend the functional longevity for chronic, reliable access to biological systems. For the most sophisticated yet delicate nervous system interfaces, elastic polymer materials including polydimethylsiloxane (PDMS), cyclic olefin copolymer elastomer (COCE), polyurethane (PU), and alginate hydrogels, have been deployed as the viscoelastic substrate for multifunctional devices which enable neural optogenetics stimulation, electrophysiological recording, drug infusion and neurotransmitter detections. Fabricating dedicated microstructures in viscoelastic devices is limited to two-dimensional architectures and heavily relies on successive and sophisticated manufacturing approaches such as lithography and micro-printing.

Thermal pulling techniques provide multiple-step scaling-down feasibility for multifunctional polymer fibers, however, this approach requires coherent parameters of the constituent materials, such as glass transition temperature (Tg), melting temperature (Tₘ) and thermal expansion coefficients (a) to be drawn into an integrated fiber. Moreover, the high-temperature process narrows the selections of available polymers for high-water-content bioelectronics. Assisted with hydrogel cross-linking as a soft material matrix, hybrid multifunction fibers permit adaptive bending stiffness for long-term sensing and neural modulation.

Aside from mechanical stiffness change in the solvated state and the desiccated state, hydrogel materials permit tunable volumetric control as the supporting scaffold. Employing hydrogel swelling behaviors in the solvated state, the expansion microscopy technique utilized hydrogel volumetric increase to enhance microimaging resolution for intact biological tissues. In contrast, hydrogel shrinking behaviors in a desiccated state have been applied to densify patterned materials in volumetric scaffold deposition and obtain nanoscale feature sizes in three dimensions. However, the hydrogel swelling and shrinking behaviors in these techniques are based on reversible polymer chains collapsing in dried state and expanding upon hydration. When applied to an aqueous in vivo environment, the shrunk hydrogels will expand and lose the miniaturized structures from the original manufacturing.

Inspired by the volumetric change which results from polymer chains' folding and expansion, the present inventors have advantageously found that by controlling the amorphous-crystalline transition in semi-crystalline hydrogels, polymer chain folding and crystallization can be tuned, and therefore the expansion of the polymer chains can be prevented, maintaining a particular hydrogel volume even under a solvated state. Hydrogel bioelectronics, miniaturized by the polymeric crystallization approaches can stably maintain their designed architectures in vivo. A significant advantage is therefore provided by the present disclosure.

Described herein is cross-linking chemistry and micro-fabrication processes to control polymeric crystalline domain growth with cross-linked polymer (e.g., polyvinyl alcohol (PVA)) hydrogels. A stable and tunable volumetric decrease of hydrogels was achieved in a hydrated state under physiological conditions (pH 6-8, 37° C.). By introducing an inorganic binder (e.g., tetraethyl orthosilicate (TEOS)) in addition to a generic cross-linker (e.g., glutaraldehyde (GA)), the hydrogel refractive index was increased while minimizing polymetric crystalline scattering. Further acidification treatment and orientated nanocrystalline growth under deformation enhanced the design of amorphous and crystalline architectures. This control of metamorphic polymers' amorphous-crystalline transition (COMPACT) strategy enabled a high (e.g., greater than 75%) diameter decrease of hydrogel fibers in the hydrated state while maintaining high stretchability (e.g., 100%-250%), low elastic modulus (e.g., 2-35 MPa, or 2 to 10 MPa), and low bending stiffness (e.g., 3 to 6.5 N/m).. The present inventors further developed core-clad hydrogel fibers with distinct RI contrast. These core-clad structured hydrogel fibers could be applied for concurrent optogenetics stimulation and photometry recordings from mouse nucleus accumbens (NAc) or ventral tedmental area (VTA) in the context of social interactions. Further taking advantage of these tunable hydrogel matrix scaffolds, conductive nanomaterials (e.g., carbon nanotubes) could be loaded into the hydrogels to provide hybrid microelectrodes. The hydrogels could further be integrated with an optical core to provide multifunctional hydrogel optrodes for in vivo electrophysiological recordings of optically triggered neural activity.

Accordingly, an aspect of the present disclosure is a miniaturized hydrogel. The miniaturized hydrogel comprises the reaction product of a hydroxyl-containing polymer, a primary crosslinker, and a secondary crosslinker.

The hydroxyl-containing polymer can include any synthetic or naturally occurring polymer having a plurality of hydroxyl-groups as pendent groups along the polymer backbone. In an aspect, the polymer has an aliphatic backbone, preferably an aliphatic hydrocarbon backbone. Exemplary hydroxyl-containing polymers can include polyvinyl alcohol, poly(hydroxypropyl methacrylate), sodium alginate, hyaluronic acid, dextran, starch, and the like, or a copolymer thereof, or a combination thereof. In an aspect, the hydroxyl-containing polymer can comprise polyvinyl alcohol or a copolymer thereof. In a specific aspect, the hydroxyl-containing polymer comprises a polyvinyl alcohol homopolymer.

The hydroxyl-containing polymer can have a number average molecular weight of greater than 10,000 grams per mole (g/mole), or greater than 25,000 grams per mole, or greater than 50,000 grams per mole, or greater than 100,000 grams per mole. For example, the hydroxyl-containing polymer can have a number average molecular weight of greater than 10,000 grams per mole to 250,000 grams per mole, or greater than 50,000 grams per mole to 200,000 grams per mole, or greater than 100,000 grams per mole to 200,000 grams per mole. Without wishing to be bound by theory, higher molecular weight polymers can provide higher stretchability of the final hydrogel product, and therefore may be preferred in certain aspects.

The primary crosslinker can be selected to provide covalent crosslinks between polymer chain. Accordingly, selection of the primary crosslinker can vary based on the hydroxyl-containing polymer. In an aspect, the primary crosslinker can comprise silicon, preferably a silicon-containing compound having at least two groups that are reactive toward hydroxyl groups (e.g., silyl ether groups). For example, the primary crosslinker can comprise an organosilicon compound. The organosilicon compound preferably comprises at least two silyl ether groups. In an aspect, the primary crosslinker can comprise a tetra(C₁₋₆ alkyl)orthosilicate, preferably tetraethyl orthosilicate.

The secondary crosslinker can be selected to further tune the optical and/or mechanical properties of the hydrogel as understood by the skilled person guided by the present disclosure. In an aspect, the secondary crosslinker can comprise an organic compound and comprises at least two functional groups which are capable of reacting with (e.g., forming covalent bonds with) the hydroxyl groups of the hydroxyl-containing polymer. In an aspect, the secondary crosslinker comprises a dialdehyde, a dicarboxylic acid, or a combination thereof. In an aspect, the secondary crosslinker can comprise the dialdehyde and the dialdehyde can be a (C₃₋₁₈ alkylene) dialdehyde, for example, glutaraldehyde. In an aspect, the secondary crosslinker can comprise the dicarboxylic acid, and the dicarboxylic acid can be a (C₃₋₆ alkylene) dicarboxylic acid. The dialdehyde or the dicarboxylic acid can each independently be substituted or unsubstituted and comprise a saturated or unsaturated alkylene group. Exemplary dicarboxylic acids can include maleic acid, sulfosuccinic acid, and the like, or a combination thereof.

While covalent crosslinks are specifically disclosed above, it will be understood that other transient crosslinks (e.g., hydrogen bonding, coordination with metals, etc.) may also be contemplated.

The reaction product of the hydroxyl-containing polymer, the primary crosslinker, and the secondary crosslinker can be present in an amount of 45 to 65 weight percent, or 50 to 60 weight percent, or 55 to 60 weight percent, each based on the total weight of the miniaturized hydrogel.

The miniaturized hydrogel further comprises water. The water may be pure water or may be, for example, an aqueous solution including other components, such as salts (e.g., buffer solutions, etc.). The water is present in the miniatured hydrogel in an amount of 35 to 55 weight percent, or 40 to 50 weight percent, or 40 to 45 weight percent, each based on the total weight of the miniaturized hydrogel.

The miniaturized hydrogel of the present disclosure is made by a method which imparts a particular structure to the hydrogel, enabling certain properties for the final hydrogel product. The hydrogel is made by a method comprising contacting the hydroxyl-containing polymer, the primary crosslinker, and the secondary crosslinker under conditions effective to provide a crosslinked hydrogel. In an aspect, the hydroxyl-containing polymer, the primary crosslinker, and the secondary crosslinker may be contacted simultaneously. In an aspect, the hydroxyl-containing polymer can be first contacted with the primary crosslinker to form a pre-gel, and then subsequently contacted with the secondary crosslinker to provide the crosslinked hydrogel. In a specific aspect, an aqueous solution of the hydroxyl-containing polymer is prepared, and the primary crosslinker can be added to the solution. The resulting mixture can be homogenized under conditions effective to provide an emulsion. A catalyst (e.g., an acid, such a HCl) can be added to effect hydrolysis of the primary crosslinker (e.g., when the primary crosslinker comprises silyl ether groups). Separately, an aqueous solution of the hydroxyl-containing polymer can be prepared and the secondary crosslinker can be added to the solution. The two solutions (i.e., a first solution comprising polymer and primary crosslinker and a second solution comprising polymer and secondary crosslinker) can be combined, for example in a weight ratio of 1.1:1 to 1:1.1, or 1.05:1 to 1:1.05, or 1:1. The precursor solution can be added to a mold, if desired for example to provide a hydrogel having a particular shape or size, and the solution can be allowed to crosslink for a desired time. The crosslinked reaction product can be demolded, if necessary, and immersed in acidic solution (e.g., aqueous HCl). The resulting acidified crosslinked hydrogel can be stretched, for example up to 200% or 100 to 200%, and dried, for example at a temperature of 50 to 100° C. for 1 to 24 hours, followed by annealing at a temperature greater than the drying temperature, for example for a time of 5 minutes to 1 hour. The recovered annealed crosslinked material can be rehydrated in aqueous solution (e.g., water) to provide the miniaturized hydrogel.

When drying under tension, the thickness of the hydrogel decreases. When rehydrated, the miniaturized hydrogel does not swell by more than 10% in any direction. When rehydrated, the miniaturized hydrogel can have a thickness that is reduced by at least 70%, or at least 75%, or at least 80% relative to the thickness of the initial hydrogel prior to drying under tension. In an aspect, the hydrogel may be in the form of a fiber. When in the form of a fiber, the diameter of the miniaturized hydrogel fiber can be reduced by at least 70%, or at least 75%, or at least 80% when rehydrated after drying under tension. When rehydrated, the miniaturized hydrogel fiber does not swell by more than 10% lengthwise, and does not substantially swell (e.g., less than 5%) in the cross direction (i.e., fiber diameter).

Advantageously, the miniaturized hydrogel comprises a plurality of nanocrystalline domains. In an aspect, the nanocrystalline domains can be present in an amount effect to provide the miniaturized hydrogel with a total crystallinity of 5 to 20%, for example 5 to less than 20%, or 5 to 18%. The nanocrystalline domains can each independently have an average size of at least 4 nanometers, for example 4 to 6 nanometers, or 4.5 to 5.5 nanometers. In an aspect, the nanocrystalline domains can have a d spacing of greater than 9 nanometers, as determined using wide-angle X-ray scattering. In an aspect, the nanocrystalline domains can have a d spacing that is greater than the d spacing of the nanocrystalline domains of the corresponding unstretched hydrogel.

The rehydrated miniaturized hydrogel can comprise 45 to 65, or 50 to 60, or 55 to 60 weight percent of the reaction product of the hydroxyl-containing polymer, the primary crosslinker, and the secondary crosslinker; and 35 to 55, or 40 to 50, or 40 to 45 weight percent water, wherein weight percent is based on the total weight of the miniaturized hydrogel. In an aspect, the primary crosslinker can be present in an amount of greater than 0 to 5 weight percent, preferably 1 to 4 weight percent, or 1 to 3 weight percent, each based on the total weight of the miniaturized hydrogel.

In some aspects, the miniaturized hydrogel can further comprise one or more additives with the proviso that they do not significantly adversely affect any of the desired properties of the miniaturized hydrogel. For example, in an aspect, the miniaturized hydrogel can further comprise a filler, for example a conductive filler. Exemplary conductive fillers can include carbon nanotubes or carbon nanofibers.

The miniaturized hydrogel can optionally be in the form of a fiber. In an advantageous feature, the diameter of the hydrogel can be selected and tuned based on the application of the miniaturized hydrogel. For example, the miniaturized hydrogel fiber can have an average diameter of 50 to 500 micrometers, for example 5 to 300 micrometers. In an aspect, miniaturized hydrogel fibers having an average diameter of 50 to 500 micrometers may be particularly well suited for neural probe application in a mouse. It will be appreciated by the skilled person that for the same application (e.g., as a neural probe) in a different species, miniaturized hydrogel fibers of a different diameter may be preferred. The diameter of the miniaturized hydrogel fiber can therefore be appropriately selected based on the application and tuned using the guidance provided herein.

The hydrogel can optionally further comprise a coating layer disposed on a surface of the hydrogel. When the hydrogel is in the form of a fiber, the coating layer can be present as an outer layer encompassing the hydrogel fiber. In an aspect, the coating layer can comprise a crosslinked polymer matrix, for example crosslinked hydroxyl-containing polymer, and a filler. When present, the hydroxyl-containing polymer of a coating layer can be the same or different as the hydroxyl-containing polymer of the miniaturized hydrogel. Preferably, the coating layer can have a refractive index that is different from the refractive index of the miniaturized hydrogel core.

The miniaturized hydrogel of the present disclosure can exhibit one or more desirable properties. For example, the miniaturized hydrogel can exhibit a stretchability of greater than 100%, preferably 100 to 300%, more preferably 100 to 250%. The miniaturized hydrogel can exhibit an elastic modulus of less than 35 MPa, or 20 to 32 MPa. The miniaturized hydrogel can exhibit a refractive index of 1.35 to 1.45 at 480 nanometers. The miniaturized hydrogel can exhibit a light transmission of greater than 95%.

The desirable combination of properties makes the miniaturized hydrogels of the present disclosure particularly well suited for various application, for example for use in implantable medical devices. An implantable medical device therefore represents another aspect of the present disclosure. In an aspect, a neural probe can comprise the miniaturized hydrogel of the present disclosure. In an aspect, a microelectrode can comprise the miniaturized hydrogel of the present disclosure. The implantable medical device (e.g., a neural probe or a microelectrode) comprises a conductive component, for example a conductive filler embedded in the miniaturized hydrogel. The neural probe or microelectrode can further include an insulating layer on the outer surface of the hydrogel fiber. The insulating layer preferably comprises a nonconductive thermoplastic polymer. The implantable medical device preferably comprises the miniaturized hydrogel in the form of an elongated fiber so as to be insertable into living tissue. The conductive miniaturized hydrogel fibers can conduct electric signals (e.g., neural spikes or electrical stimulation) between the tip of the of the probe inserted into a tissue and the device's or probe's proximal end. The implantable medical device may also comprise a miniaturized hydrogel in the form of an elongated fiber capable of guiding light between the tip and the proximal end.

The miniaturized hydrogel of the present disclosure is also believed to be novel independent from the method of manufacturing. Accordingly, a miniaturized hydrogel represents another aspect of the present disclosure. For example, a miniaturized hydrogel can comprise 45 to 65, or 50 to 60, or 55 to 60 weight percent of a reaction product of a hydroxyl-containing polymer, a primary crosslinker, and a secondary crosslinker; and 35 to 55, or 40 to 50, or 40 to 45 weight percent water; wherein weight percent is based on the total weight of the miniaturized hydrogel. The hydrogel can comprise a plurality of nanocrystalline domains, wherein the nanocrystalline domains are present in an amount effective to provide the hydrogel with a total crystallinity of 5 to 20%. The miniaturized hydrogel exhibits one or more of: a stretchability of greater than 100%, preferably 100 to 300%, more preferably 100 to 250%; or an elastic modulus of less than 35 MPa, or 20 to 32 MPa or 1 to 35 MPa, or 1 to 15 MPa, or 1 to 10 MPa; or a refractive index of 1.35 to 1.45 at 480 nanometers; or a light transmission of greater than 95%.

Another aspect of the present disclosure is a method of interfacing with neural tissue. The method comprises inserting a first end of a neural probe into the neural tissue, wherein the neural probe comprises the miniaturized hydrogel of the present disclosure. The method further comprises stimulating the neural tissue at the first end of the neural probe with an electromagnetic signal.

Another aspect of the present disclosure is a method of electrically detecting a transfer of charge between or within cells in living tissue. The method includes providing a microelectrode comprising the miniaturized hydrogel of the present disclosure in intimate contact with tissue capable of transferring electronic charge. The miniaturized hydrogel preferably includes a conductive filler such as carbon nanotubes to provide a working electrode. The microelectrode can transmit or receive an electrical signal between the conductive miniaturized hydrogel and the biological component. The method also includes electrically connecting the microelectrode to a power source. The method further includes applying a voltage or current across the conductive miniaturized hydrogel.

As described herein, the method for the manufacture of the miniaturized hydrogel of the present disclosure imparts a certain semi-crystalline structure to the miniaturized hydrogel, which can further lead to the desirable properties. Accordingly, a method for the manufacture of a miniaturized hydrogel represents another aspect of the present disclosure.

The method for the manufacture of a miniaturized hydrogel comprises contacting a hydroxyl-containing polymer, a primary crosslinker, and a secondary crosslinker under conditions effective to provide a crosslinked hydrogel; acidifying the crosslinked hydrogel; drying the crosslinked hydrogel under tension; and rehydrating the dried hydrogel to provide the miniaturized hydrogel. An exemplary method is further described in the working examples below.

This disclosure is further illustrated by the following examples, which are non-limiting.

### EXAMPLES

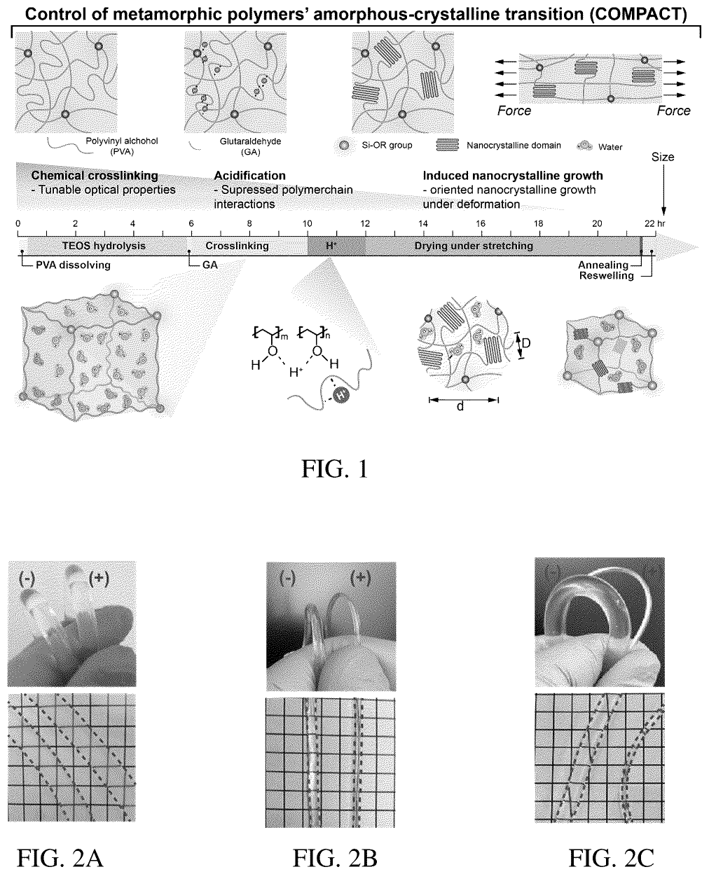

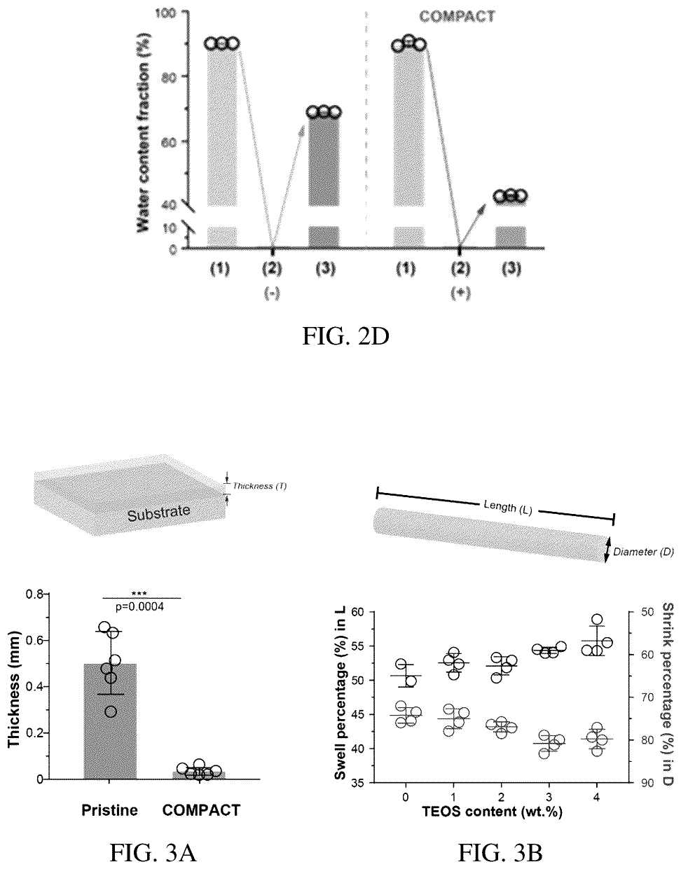

To explore PVA hydrogels and controllable miniaturization properties while preserving the advantages of superior optical properties, fatigue resistance, and biocompatibility, new hydrogels were designed using fabrication approaches by control of metamorphic polymers' amorphous-crystalline transition (COMPACT) from such aspects: (i) polymer chains folding and immobilization with multiple cross-linkers, (ii) intervention on intermolecular chain interactions in the hydrogel matrix, (iii) orienting nanocrystalline domains growth. The COMPACT strategy was implemented following three major procedures to control individual polymer chain folding, polymer chain network interactions and nanocrystalline growth. First the inorganic binder tetraethyl orthosilicate (TEOS) was introduced to a PVA solution through homogenization (FIG. 1), followed by addition of a generic cross-linker, glutaraldehyde (GA). Without wishing to be bound by theory, it is believed that the combination of the two types of cross-linkers allows the control of polymer chain mobility via covalent bonding and parallel tuning of hydrogels' refractive index. The cross-linked hydrogels were acidified to promote hydrogen bond generation among polymer chains. External mechanical stretching was applied to the fully acidified hydrogels and maintained during the desiccating process. After the removal of water from hydrogels, high-temperature (e.g., 100° C.) annealing was employed to further promote the growth and orientation of the nanocrystalline domains. To test whether polymeric nanocrystalline domains created through COMPACT strategy can preserve hydrogel volumetric shrinking under hydrated status, the dimensions and water fractions of cross-linked hydrogels under pristine, desiccated, and re-hydrated states was examined, as shown in FIG. 2A-2C.

Fiber-shaped hydrogels were prepared via molding and extrusion methods. At the pristine and desiccated states (FIGS. 2A and 2B), the two hydrogel fibers with TEOS-GA cross-linking (COMPACT+) and GA cross-linking (COMPACT−) exhibited comparable geometries and water fractions (FIGS. 2A, 2B, and 2D), however, only the TEOS-GA cross-linked PVA hydrogel fiber with acidification and mechanical stretching maintained the reduced diameters in the re-hydrated state (FIGS. 2C and 2D).

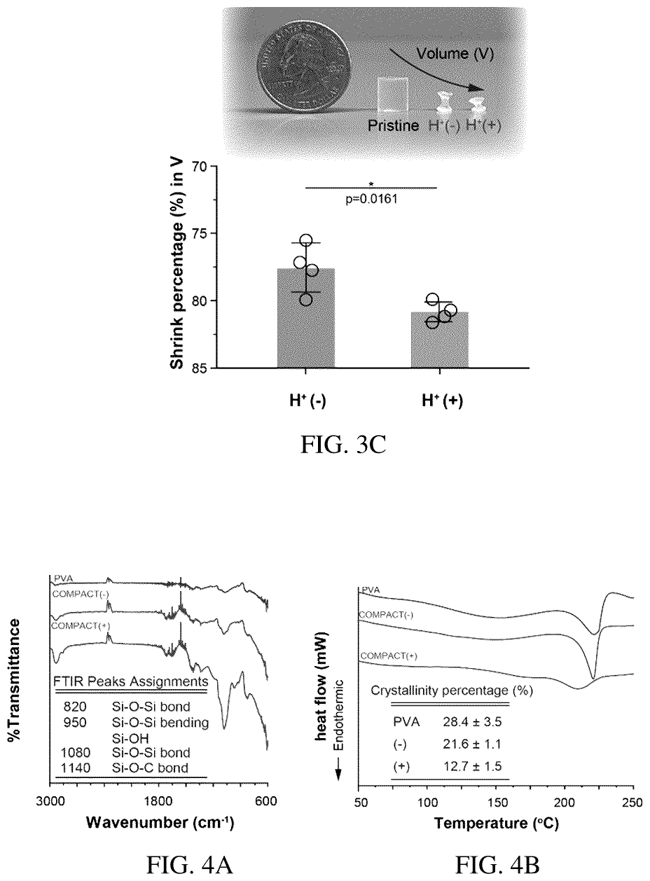

After confirming that hydrogels retained perpetual shrinking behaviors in re-hydrated state with COMPACT treatment, size reduction dependence on the materials' geometries and external constraints was tested. Thin-film, fiber-shaped, and bulk hydrogels were prepared, and the changes of COMPACT hydrogel film thickness (T), fiber diameter (D) and volume (V) were examined, as shown in FIG. 3A. TEOS-GA cross-linked PVA hydrogel thin films with acidification treatment exhibited a thickness reduction ratio of 93.4±3.6% (pristine thickness: 501±134 μm; re-hydrated thickness: 33±18 μm) under optical microscopy examination (FIG. 3A, bottom). TEOS-GA cross-linked PVA hydrogel fibers, with applied acidification and mechanical strain (200%) treatments, reached the maximum diameter shrinking ratio of 79.7±2.3%, by increasing the content of the TEOS cross-linker (FIG. 3B). In three-dimensional free shrinking structures, 80.9±0.7% volumetric shrinking in acidified TEOS-GA cross-linked cylinders was observed, as compared to pristine ones (FIG. 3C).

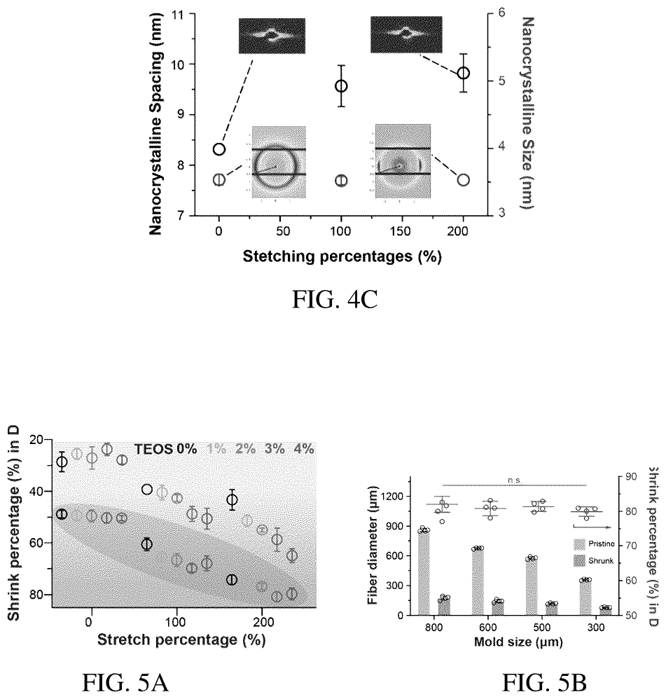

Mechanisms of the sustained hydrogel volume decrease was evaluated. Fourier transform infrared spectroscopy (FTIR) results indicated covalent bonds (Si—O—Si and Si—OH) generated in COMPACT hydrogel network (FIG. 4A). The new Si—O—Si (1080 cm⁻¹) and Si—OH (950 cm⁻¹) bonds came from hydrolyzed TEOS Si—OR groups' reactions with the hydroxyl groups on PVA chains. The generic cross-linker GA reactions were confirmed by the observation of C═O bond (1740 cm⁻¹) and the Si—O—C bond (1140 cm⁻¹) from the reaction with TEOS Si—OR groups. Besides confirming covalent bonds generated among hydrogel polymer chains, differential scanning calorimetry (DSC) results exhibited the change of polymer chain interactions and polymeric crystallinity after COMPACT treatment. Undissolved PVA powders showed 28.4±3.5% crystallinity (FIG. 4B), similar to the reported crystallinity percentage of semi-crystalline PVA polymers. GA-cross-linked PVA hydrogels exhibited 21.6±1.1% crystallinity while the additional TEOS cross-linking, and acidification suppressed the polymer chain folding to form crystalline domains (crystallinity: 12.7±1.5%). The nanocrystalline domains and orientation was further analyzed with X-ray scattering techniques. The size of PVA nanocrystals was measured as 3.5±0.1 nm while the nano-crystalline spacing increased from 8.32±0.08 nm to 9.83±0.38 nm after 200% axial stretching (FIG. 4C). Wide-angle X-ray scattering (WAXS) 2D patterns suggested that the lamellae crystal domains were re-oriented along the axial stretching direction (FIG. 4C).

In COMPACT hydrogels, chemical cross-linking and acidification treatment both contribute to the retained volumetric decrease upon re-hydration while mechanical deformation induces the orientated nanocrystalline growth. An increased number of chemical cross-linkers, TEOS (0-4 wt %), enhanced the anchoring of amorphous PVA chains through covalent cross-linking and prevented swelling in the hydrated state. Under the same cross-linking degree, acidification treatment granted polymer chains enhanced interactions and suppressed the folding of polymer chains to form crystalline. Nanocrystalline domains maintained the nanoscale size (˜3.5 nm) without compromising the light transmittance in the visible range. Axial mechanical deformation introduced tensile stress to re-orientate polymer chain alignment and created anisotropic nanostructures, which enabled hydrogel fibers having a desired decrease in diameter while causing a minimal effect on crystallinity degree or nanocrystalline size.

Controllable hydrogel shrinking through cross-linking and polymer chain crystallization process provides an effective methodology to miniaturize hydrogel bioelectronics, especially for the application in vivo. The approaches to regulating polymer chain folding and interactions can be extended to other semi-crystalline polymers. Without affecting the nanocrystalline size, the mechanical stretching method offers a straightforward way to create anisotropic orientations of polymeric nanostructures.

Based on COMPACT-enabled hydrated hydrogels size reduction, this methodology was expanded to develop a series of hydrogel fibers with controlled diameters and tunable optical and mechanical properties for biomedical use. A rational and comprehensive shrinking diagram was mapped by varying the content of inorganic cross-linker (TEOS), acidification, and external mechanical stretching (FIG. 5A). Generally, increasing cross-linking density with more cross-linkers led to less ductile polymer chains with reduced dimension upon hydration. Acidification treatment dramatically boosted shrinking percentages across different cross-linking densities while mechanical static stretching further enhanced the decrease of hydrogel fibers in diameters (79.7±2.3%).

To fit COMPACT into a practical molding-extrusion fabrication process, a series of hydrogel fibers were made with different sizes of silicone molds (FIG. 5B). All COMPACT hydrogel fibers reached reduced diameters of more than 79%, which is consistent with the shrinking diagram. For example, using 300 μm silicone molds (inner diameter, ID), thin hydrogel fibers were fabricated with diameters of 80±4 μm.

Considering fiber optic in vivo applications, the optical, mechanical and biocompatible properties of COMPACT hydrogel fibers were examined. To ensure efficient light transmission for optical stimulation and recordings, two parameters of the hydrogel fiber core were considered: refractive index (RI) and light transmittance. It was observed that hydrogels' refractive indices can be tuned by increasing TEOS contents. COMPACT hydrogels with 0 wt. % to 4 wt. % TEOS contents exhibited refractive indices ranging from 1.48 to 1.60 in desiccated state (FIG. 5C) and 1.37 to 1.40 in the hydrated state. Although all the transmittance remained above 96%, increasing TEOS content also led to decreased transmittance (FIG. 5C), and increased autofluorescence (17.8% increase of 4 wt. % TEOS hydrogels compared to 0 wt. % TEOS hydrogels, excitation wavelength: 485 nm, excitation peak: 520 nm). A preferred TEOS content was chosen as 3 wt. %, which resulted in hydrogels with 1.54±0.01 of refractive index, >96% of transmittance (for 0.15±0.02 mm thick membranes), and 6.13±0.16 relative fluorescent units (RFU)/mm of autofluorescence (for 0.15±0.02 mm thick membranes).

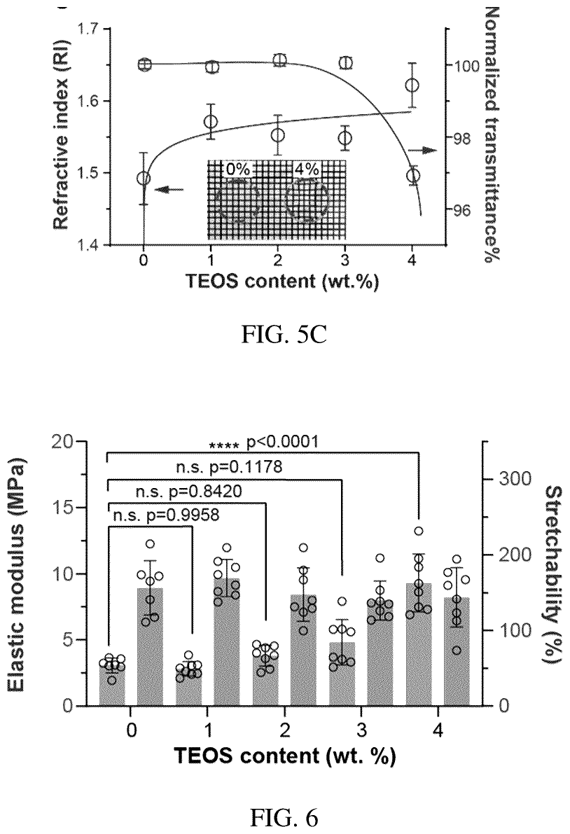

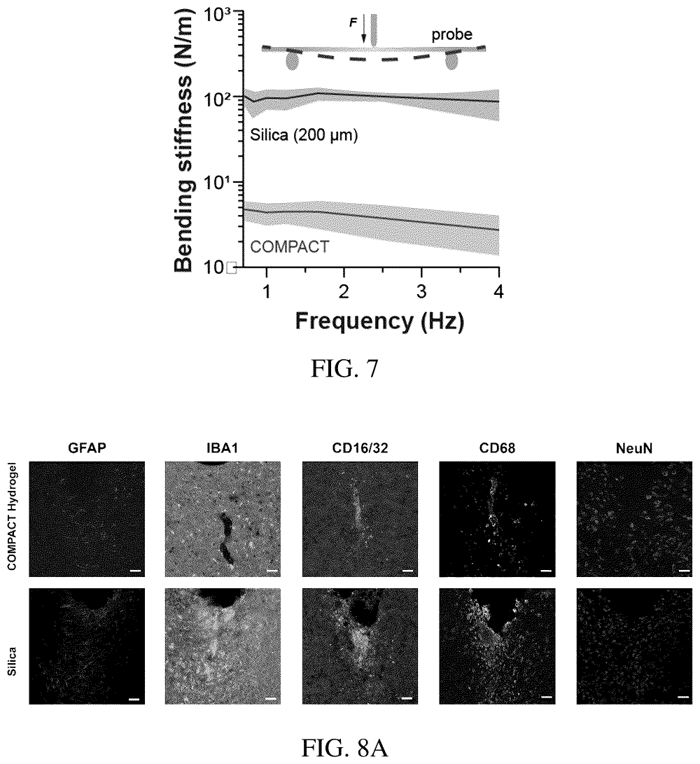

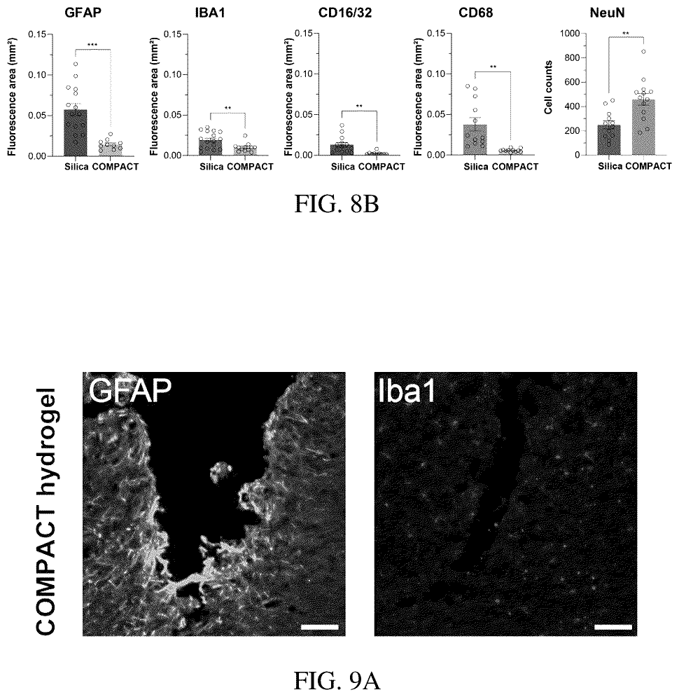

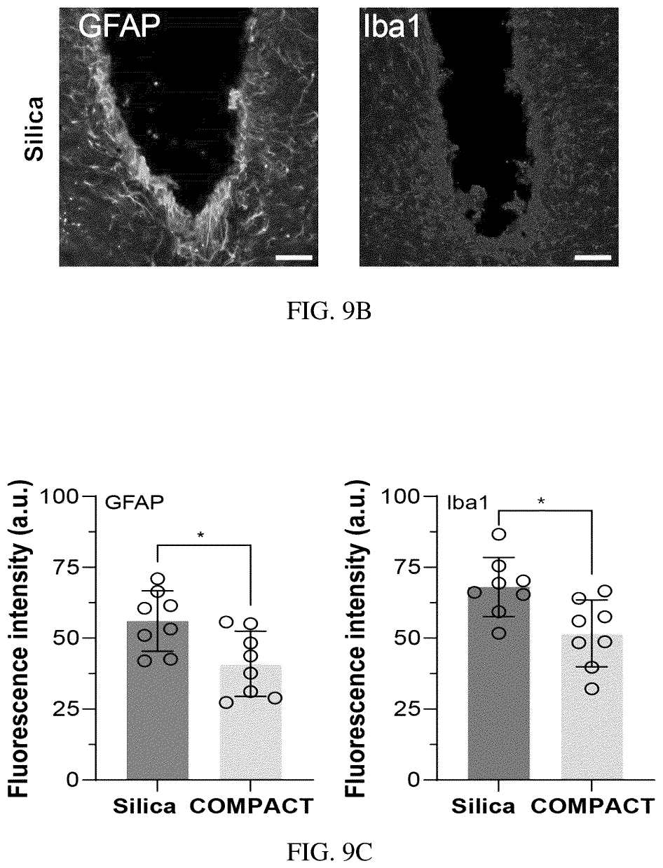

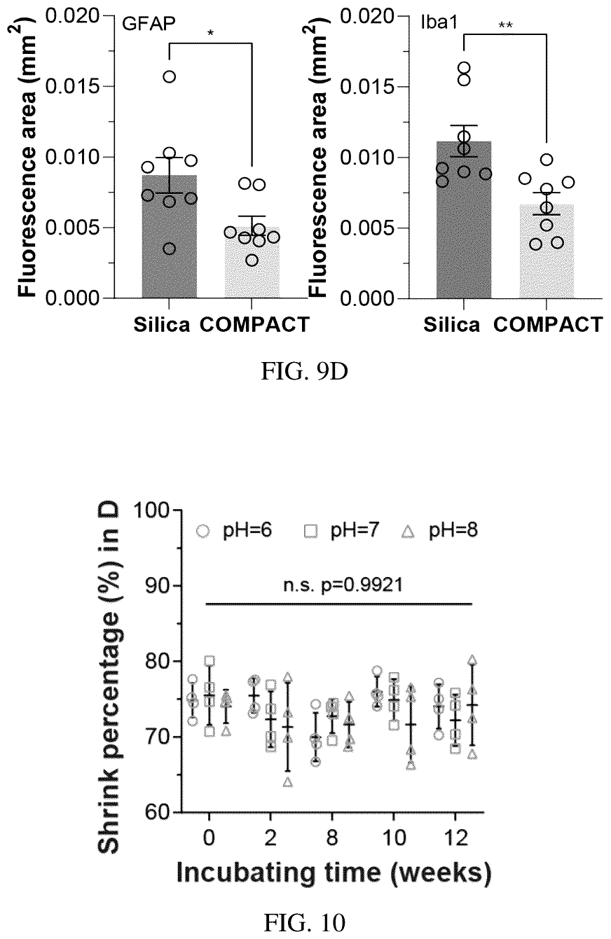

To mimic in vivo working conditions, mechanical properties of COMPACT hydrogels in the hydrated state were examined. The COMPACT hydrogel fibers exhibited relatively low elastic moduli while maintaining high stretchability, as shown in FIG. 6. An exemplary COMPACT hydrogel fiber (3 wt. % TEOS, 12 mM HCl acidification treatment and 200% stretching, diameter: 227±18 μm) exhibited an elastic modulus of 4.8±1.7 MPa and stretchability of 139.4±26.0%. When fiber-shaped neural probes are inserted into brain tissues, their axial bending stiffness serves as an important mechanical parameter under brain micromotions. Compared to silica fibers (˜20 GPa elastic modulus) and polymer fibers (˜1 GPa elastic modulus), COMPACT hydrogel fibers offer improved mechanical matching to the nervous tissues (1-4 kPa) and much lower axial bending stiffness, as shown in FIG. 7 to achieve less neural tissue damage from micro-motion involved in vivo studies. Initial evaluations focused on the immune response of brain tissues 14 days post-implantation. A reduced presence of astrocytes, microglial accumulations, activated macrophages, and immunoglobulin G was observed around the sites of hydrogel fiber implantation compared to the stiffer silica fibers, as shown in FIGS. 8A and 8B. Subsequently, the chronic immune response after 30 days was assessed and a lower incidence of astrocyte and microglial formation around the hydrogel fiber sites relative to those with silica fibers was noted, as shown in FIG. 9A-9D.

COMPACT hydrogels were tested to examine whether crystalline-enabled size reduction can overcome the intrinsic hydrogel swelling behaviors upon hydration and maintain structural stability in vivo. COMPACT hydrogel fibers were incubated in ex vivo physiological conditions (pH: 6-8, 37° C., saline solution) and fiber dimensions were monitored over time.

The shrinking percentage was maintained above 74% for over 3 months (FIG. 10). Unlike other established approaches to shrink hydrogels via desiccation, where collapse of polymer chain during drying leads to reversible swelling upon hydration, COMPACT hydrogels' polymetric nanocrystalline and enhanced interpolymer chain interactions maintained stable folding in the hydrated state and therefore permit retained volumetric size reduction. Over 3 months of incubations under physiological temperature and osmolarity, the shrunk COMPACT hydrogel fibers maintained the designed diameters within less than 1% variance, which illustrates hydrogel bioelectronics' volumetric stability of their miniaturized size in vivo. In contrast, COMPACT hydrogel fibers incubated at PVA dissolution temperature (100° C.) in water for several hours resumed their pristine swollen size; this volume reversion demonstrates the crystalline impact on size reduction through control of local free volume in hydrogel matrices. This crystalline-dominated hydrogel miniaturization phenomenon can be extended to other semi-crystalline polymers at different material interfaces, where volumetric stability is important, such as the proton-exchange membrane in packed fuel cells.

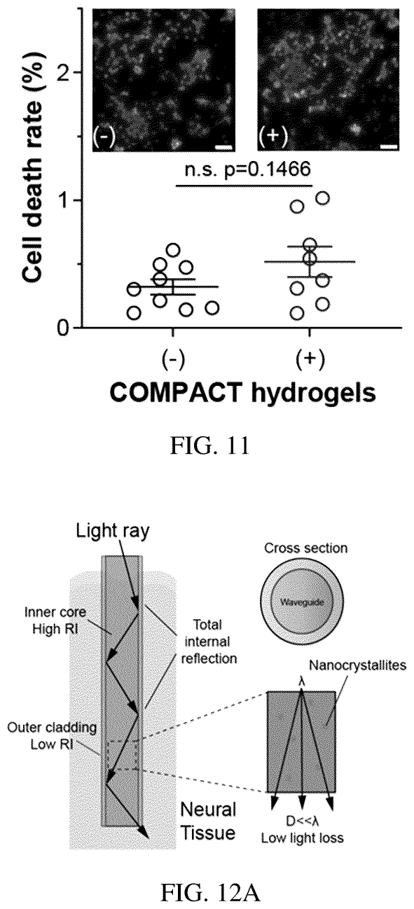

Cytotoxicity tests with human embryonic kidney cells (HEK293) exhibited no significant cell death in the presence of COMPACT hydrogels, as shown in FIG. 11.

COMPACT hydrogels were fabricated into step-index optical fibers. Increased RI contrast between optical core and cladding layers ensures light transmission and the consequent photodetection sensitivity. Based on tunable refractive indices of COMPACT hydrogels, step-index hydrogel fibers with high-RI core (n_{core}=1.40) and low-RI cladding (n_{cladding}=1.34) were designed.

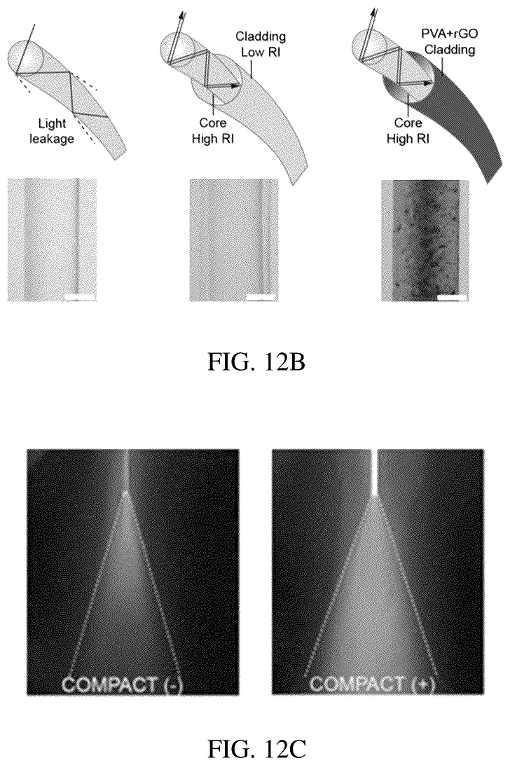

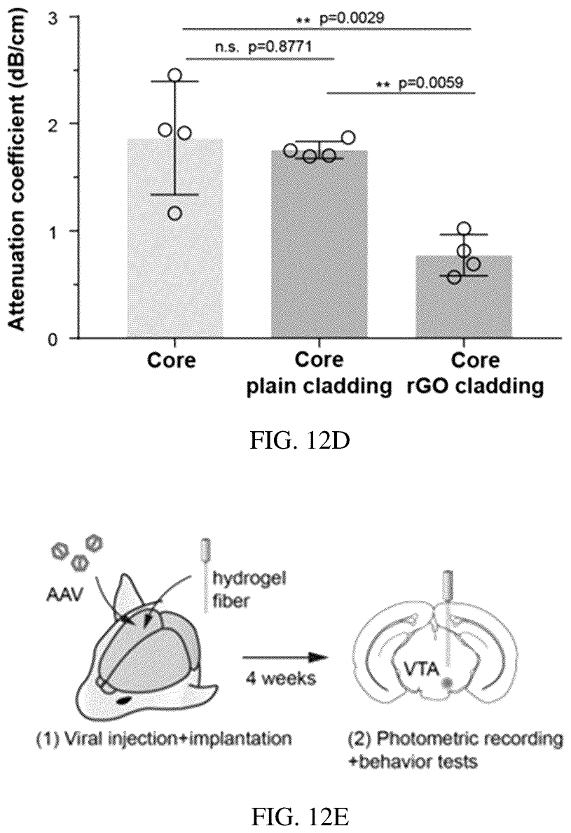

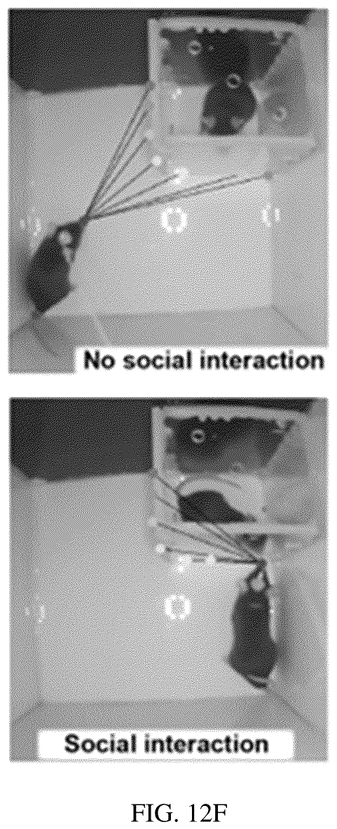

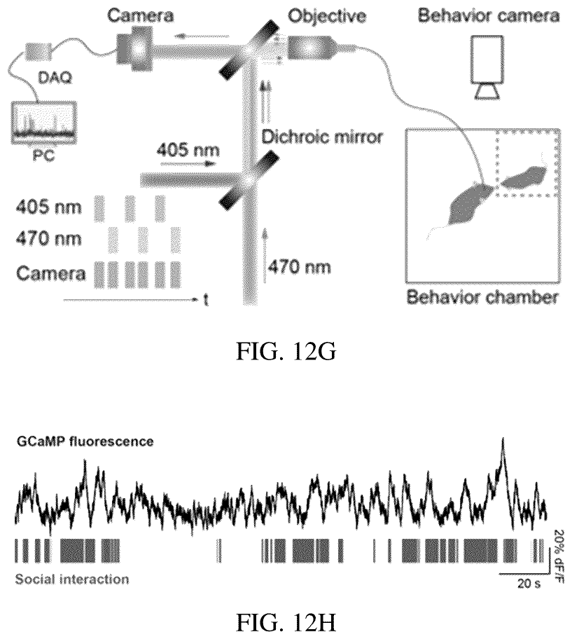

Hydrogel fibers were connected to a silica segment embedded in an optical ferrule with a strong connection while preventing directly exposed hydrogels dehydration out of tissues and light loss. The function of RI-contrasting core-clad structures was validated by comparing the light transmission between bare core fibers, step-index fibers with plain cladding and those with light-protective cladding. As shown in FIG. 12D, the bare core fibers (diameter of 329±17 μm) exhibited a relatively high attenuation (1.87±0.53 dB/cm) while introducing a thin low-RI cladding layer (thickness of 84±4 μm on the surface of 372±10 μm cores, n_{cladding}=1.34) decreased the light transmission attenuation to 1.75±0.08 dB/cm. A representative light-absorption nanomaterial (cite), reduced graphene oxide (rGO) was loaded into low-RI cladding to further protect light leakage from fibers' lateral surface and consequently reduced the light attenuation to 0.94±0.25 dB/cm (core 339±35 μm, cladding: 36±11 μm of 5 wt. % PVA with 0.21 wt. % rGO), as shown in FIG. 12D.

To validate the functionality for in vivo optical stimulation and recording, COMPACT hydrogel fibers were tested with concurrent optogenetics and fiber photometry in the context of mouse behaviors. Activation of ventral tegmental area (VTA) region has been studied with various techniques, including optogenetics, chemogenetics and electrical stimulation, related to social behaviors in mice. As a proof-of-concept application of COMPACT optical fibers, photometric recording of mouse VTA with social behavior tests were selected. COMPACT optical fibers (580±35 μm) were unilaterally implanted in VTA after injecting a adeno-associated virus (AAV) containing genetically encoded calcium indicator (hSyn::GCaMP6s). A home-built fiber photometry system associated with was used to collect GCaMP fluorescent change to reflect neural activity. The stiffness change of hydrogel fibers from a dried state (stiff) to a hydrated state (soft) was utilized and implanted the hydrogel fiber in the dried state with calibrated coordinates. A fiber photometry system (wavelengths: λ_{isosbestic point}=405 nm, λ_{excitation}=470 nm, λₑₘᵢₛₛᵢₒₙ=510 nm) was used to collect GCaMP fluorescent changes as proxies to reflect the neural activity. After an incubation period of 4 weeks with confirmed virus expression in the VTA, mice were subjected to a social preference test with photometric recordings Mouse social preference tests were recorded and further analyzed with DeepLabCut and custom-developed MatLab algorithm. Increased fluorescent intensity of GCaMP was observed and was correlated with mouse social interaction epochs. Linking the neural activities at the cellular level to system neuroscience behavioral assessment provides important tools to discover causal relationship of neural circuits and behaviors for neuroscience studies.

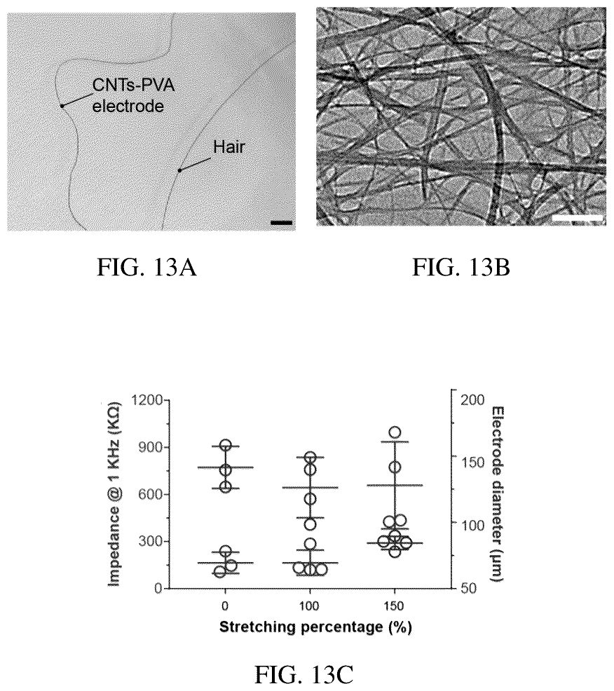

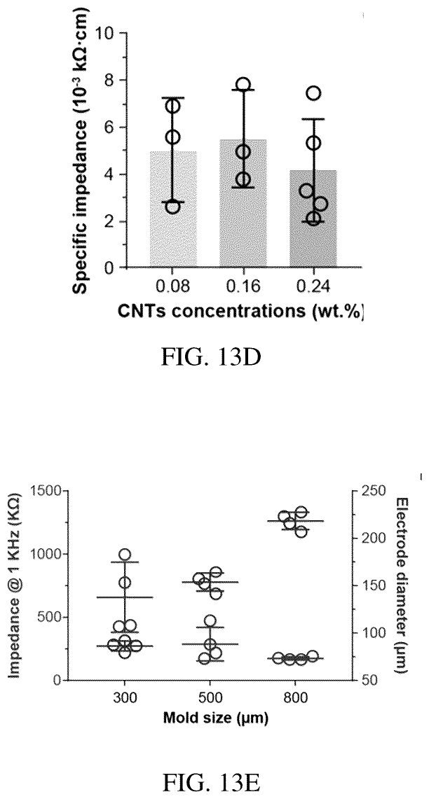

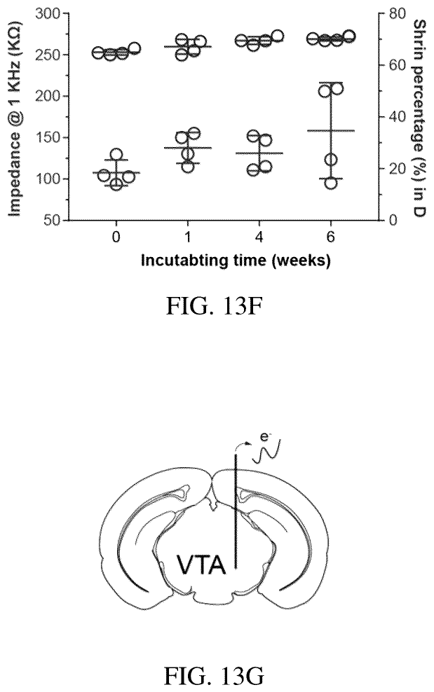

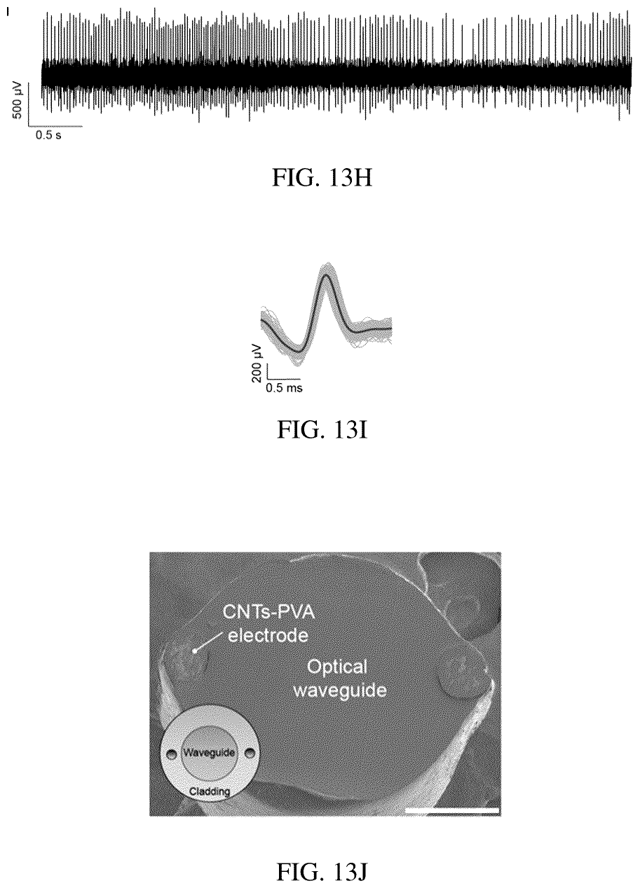

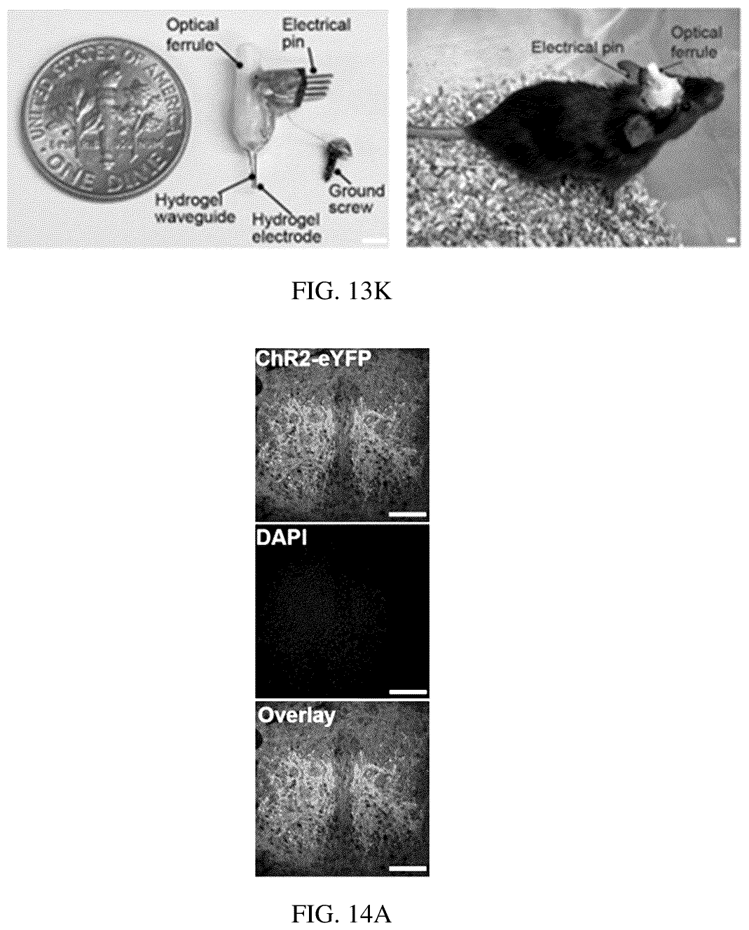

Hydrogel matrices can support various nanoscale materials to extend the functionalities while maintaining the desired mechanical properties. To enrich hydrogel neural probe modality for electrical recordings, conductive carbon nanotubes (CNTs, 12±6 nm diameter) were incorporated into PVA hydrogel scaffolds during hydrogel cross-linking (FIGS. 13A and 13B). Acidification and mechanical stretching facilitated CNT plaited into polymer matrices and ensured entangled with PVA chains and consequently augmented electrical conductivity as a percolated network. CNTs-PVA hydrogel electrodes (86±5 μm diameter) exhibited stable impedances of 658±277 kΩ at 1 kHz (saline, 25° C.) and impedance was tunable with designed mold sizes and CNT loadings, as shown in FIG. 13C-13E. CNTs-PVA hydrogel electrodes were insulated with a viscoelastic coating of styrene-ethylene-butylene-styrene (SEBS). To verify the stability of CNTs-PVA hydrogel electrodes, they were incubated in PBS solution and impedance was characterized over 6 weeks. The results are shown in FIG. 13F, and no significant increase of impedance at 1 kHz was found.

CNT-PVA hydrogel electrodes were employed for electromyographic (EMG) recordings of mouse hindlimb muscles in response to the pulsed blue light illumination. CNT-PVA hydrogel electrodes detected hindlimb muscle electrical signals upon transdermal optical stimulation (wavelength λ=470 nm, 200 mW/mm², 0.5 Hz, pulse width 50 ms) in Thy1::ChR2-EYFP mice, which express photo-excitatory opsin, Channelrhodopsin 2 (ChR2), in nervous system. Instead of recording collective electrical response from muscles, CNTs-PVA hydrogel electrodes were then implanted in mouse VTA to record neuron spontaneous spiking activity in anesthetized wild-type mice under continuous isoflurane (see FIG. 13G-13I). A bandpass filter of 300-3000 Hz was applied to detect spiking activity, and one distinct cluster of spikes of principal component analysis (PCA) was observed. The signal-to-noise ratio (SNR) of these spiking activities was approximately 3.73 with repeatable waveforms.

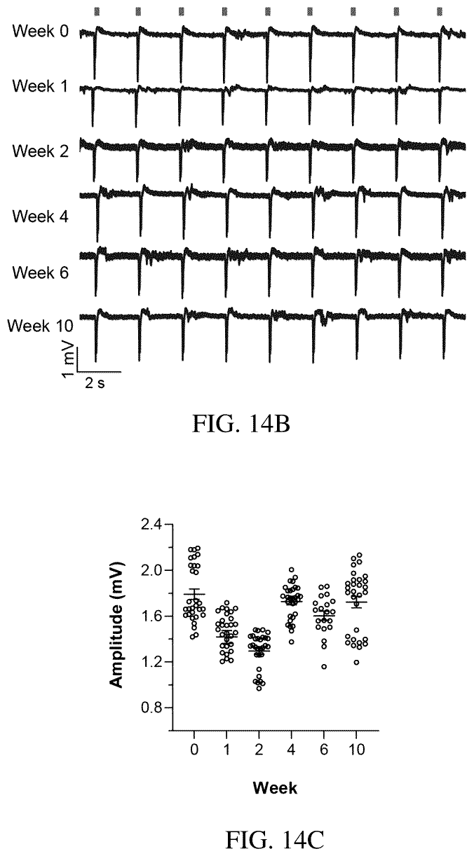

When extending hydrogel miniaturization from bulk materials to interfaces, the COMPACT strategy offers new revenue for multiple components integration. Since RI-distinct core-clad structures ensure light transmission in optical cores, two CNT-PVA electrodes were introduced into the cladding layers with a COMPACT hydrogel core. This hydrogel optrode is designed to enable optical modulation with concurrent electrophysiological recording. In Thy1::ChR2-EYFP mice, blue light pulses (λ=470 nm, 0.5 Hz, pulse width 50 ms, 10 mW/mm²), delivered through the hydrogel optical core, consistently activated Channelrhodopsin 2-expressing neurons in VTA while the neural electrical signals were collected through CNT-PVA electrodes. The optical evoked potentials were repeatedly captured with correlation with the onset of light stimulation over 10 weeks post-implantation, shown in FIGS. 14B and 14C.

In this study, a set of hydrogel cross-linking chemistry and fiber-shaped device microfabrication approaches were developed through a bottom-up strategy of tuning polymers' amorphous-crystalline transition for hydrogel bioelectronics miniaturization and integration. The COMPACT strategy provides an accessible, scalable, and controllable fabrication method for microstructured hydrogel fibers as small as 80 μm with consistently low asperity. These hydrogel matrices provide a platform for functionally augmented interfaces through loadings of additional nanomaterials. COMPACT hydrogels can be further designed into step-index optical probes and optrodes which are well-suited for neural modulation and recordings concurrent with behavioral assays in mice.

Distinguished from the reported hydrogel volumetric shrinking through polymer chain collapse upon desiccation, in which polymer chains will stretch and hydrogels consequently swell again upon hydration, COMPACT hydrogels' polymetric nanocrystalline and enhanced interpolymer chain interactions maintained stable folding in the hydrated state and therefore permit persistent volumetric size reduction. Over 3 months of incubations under physiological temperature and osmolarity, the shrunk COMPACT hydrogel fibers maintained the designed diameters within less 1% variance, which illustrates COMPACT bioelectronics' volumetric stability of their miniaturized size in vivo. In contrast, COMPACT hydrogel fibers incubated at PVA dissolution temperature (100° C.) in water for several hours resumed their pristine swollen size; this volume reversion demonstrates the crystalline impact on size reduction through control of local free volume in hydrogel matrices. This crystalline-dominated hydrogel miniaturization phenomenon can be extended to other semi-crystalline polymers at different material interfaces, where volumetric stability is important, such as the proton-exchange membrane in packed fuel cells.

Controllable hydrogel shrinking provides an effective methodology for miniaturization and integration for neural probe fabrication. The molding and extrusion approaches offer a series of precisely controlled hydrogel fiber diameters with structural homogeneity and low surface asperity to avoid diffuse reflection at the hydrogel interfaces. Although the mold sizes are commercially limited, COMPACT procedures, including regulating polymer and crosslinker constituent content and fiber extensions can expand the range of available fiber sizes. Successive rounds of molding with strong polymer chain infiltration at the interfaces enable the design of multimodal microstructures, including core-clad (30-80 μm) in step-index optical probes and electrodes integration in the cladding layer of optrodes.

COMPACT strategy is generalizable for soft and stretchable bioelectronics. Polymer matrices provide sufficient free volume for water access as well as nanomaterials incorporation. High aspect-ratio nanomaterials, such as silver nanowires and carbon nanotubes, can be effectively entangled with polymer chains through cross-linking and condensation during acidification and stretching. This procedure augments electrical conductivity while maintaining viscoelasticity. The colloidal stability of nanomaterials in viscous polymer precursor solutions is important to create a homogeneous composite after cross-linking to prevent phase separation and ensure stable electrical conductivity.

Compared to other viscoelastic bioelectronics fabrication approaches, such as lithography and micro-printing, COMPACT technique offers scalable and efficient multimodal hydrogel fibers manufacturing without the need for expensive and sophisticated facilities. COMPACT multifunctional neural probes have been demonstrated for bi-directional optical interrogation concomitant with mouse social behaviors and electrical recordings of light-triggered neural activity in mice. Extended functionalities, such as drug or viral vector delivery, can be further achieved by integrating additional microfluidic channels in the cladding layer without affecting light transmission efficiency in the optical core. COMPACT multifunctional neural probes involve independent components alignment and miniaturization steps, which potentiates the integration of multiple components with various lengths to target multiple depths of tissue within single-step implantation. This adaptability will increase the density of functional interfaces and overcome the traditional limitation of fiber-shaped neural probes with single-target interfaces at the tip.

Control over semi-crystalline polymers' amorphous-crystalline transition creates a direct fabrication methodology for viscoelastic materials. Extending it to the manufacture of sophisticated optoelectronic devices, the COMPACT strategy imparts a generalizable and modular platform for viscoelastic bioelectronics' miniaturization and integration, which consequently enables multimodal interrogation of complex biological systems.

Experimental details follow.

Hydrogel synthesis. Tetraethyl orthosilicate (TEOS, Sigma-Aldrich 86578, 99%), hydrochloric acid (HCl, Sigma-Aldrich, 258148, 37%), glutaraldehyde solution (GA, Sigma-Aldrich G6257, 25% in water), and PVA (average molecular weight 146,000 to 186,000 Da, 99+% hydrolyzed, Sigma-Aldrich) were purchased from Sigma-Aldrich. MilliQ water (18 MΩ·CM at 25° C.) was used throughout the experiments. PVA (10 wt. %) was dissolved in MilliQ water and stirred in a water bath at 100° C. for at least 4 hours until a clean and transparent pre-solution was obtained. The in-situ hydrolysis of TEOS was conducted with HCl as the catalyst in PVA pre-solutions (Molar ratio of TEOS: HCl: H2O=x: 4: y, where x was between 1 to 4, and y started from 4 to 16). TEOS solutions ranging from 2 wt. % to 8 wt. % were added into dissolved PVA pre-solution followed by two different level homogenizations. HCl and MilliQ water were mixed in the molar ratio of 4: y, where y was in the range of 4 to 16. A portable homogenizer was used to apply homogenization to PVA-TEOS solutions for at least 1 min to disperse oil-phase TEOS in water-phase PVA until emulsion solution was observed. A high-speed homogenizer (FSH2A lab) was used for further homogenization. The diluted HCl solutions were added into the PVA-TEOS emulsions drop by drop while homogenizing at 12000 rpm. The mixed solutions were stirred in the water bath at 100° C. for 1 hour until transparent solutions were seen followed by another 12 hours of stirring at 60° C. The compositions of all precursor solutions are provided in Table 1.

TABLE 1

| TEOS:HCl:H₂O | TEOS wt. % in PVA | HCl wt. % | PVA wt. % |
|---|---|---|---|
| (molar ratio) | pre-solutions | in solutions | in solutions |
| 1:4:4 | 2 | 0.014 | 10 |
| 2:4:8 | 4 | 0.014 | 10 |
| 3:4:12 | 6 | 0.014 | 10 |
| 4:4:16 | 8 | 0.014 | 10 |

COMPACT hydrogel fiber fabrication. The precursor solution was infused into a silicone mold (300˜800 μm) and allowed to cross-link into a fiber shape at room temperature (RT) for 4 hours. The silicone mold with cross-linked PVA hydrogel was immersed in Dichloromethane (DCM) for 5 minutes to induce the swelling of the silicone mold. Cross-linked PVA hydrogel fiber was eluted from the tubing and immersed in 12 mM HCl solutions for 2 hours. The acidified PVA hydrogel fiber was stretched and dried at 60° C. for 12 hours following by annealing at 100° C. for 20 minutes. COMPACT hydrogel fiber was achieved through swelling the annealed PVA hydrogel fiber in water.

Optical hydrogel probe fabrication. Step-index multimode fiber (core diameter 400 μm, NA 0.5, Thorlabs FP400URT), ceramic optic ferrule (bore diameter 400 μm, Thorlabs CFX440-10), fiber stripper (Micro-strip, Micro Electronics, Inc), and polish kit (Thorlabs D50-F, NRS913A, and CTG913) were supplied by Thorlabs, Inc. A silica fiber was prepared with the fiber stripping tool to get rid of the protective coating. The silica fiber was separated by a diamond cutter into 13 mm short fibers. These short fibers were inserted and extruded out at one end of the ferrule with a length of 2.5 mm before fixing with the adhesive (EccoBond F, Loctite). Both ends of the silica fibers in the ferrules were polished by the polish kit. All the silica fibers and ferrules were subjected to the examination of light transmission by coupling with a 470 nm blue light-emitting diode (LED) light (Thorlabs M470F3) after polishing. The extruded silica fibers were treated by 2M sodium hydroxide solution for 12 hours followed by additional treatment by chloroform (Sigma-Aldrich, 472476) for 30 mins until the plastic coatings on the silica fibers were removed. A thin layer of 10 wt. % PVA was coated on extruded silica fibers via dip coating. The PVA-coated silica fibers were dried at 60° C. for 12 hours and annealed at 100° C. for 2 hours.

A vacuum planetary mixer (Musashi ARV-310) was used for the mixing and degassing of all solutions. 100 μL GA was added into 10 g of 10 wt. % PVA pre-solution and mixed in the mixer at 2000 rpm for 1 minute with a vacuum level at 16 kPa. 10 g of pre-made PVA-TEOS solution was degassed at 2000 rpm and 16 kPa for 1 minute. The above two solutions were combined on a 1:1 ratio and mixed at 2000 rpm and 16 kPa for 1 minute. The mixed PVA-TEOS-GA solution was infused into silicone tubes (80 mm in length with different inner diameters, McMaster-Carr), and the optic ferrules were inserted into the silicone tubes with the silica fiber end connecting with the PVA mixture. Dichloromethane (DCM, Sigma-Aldrich 270997, 99.8%) was used to demold PVA-TEOS-GA fibers after curing at room temperature for 4 hours. The fibers were washed with a large amount of water to remove residual chemicals for two days. Ferrule-connected fibers were dried in the air at room temperature for 12 hours and annealed at 100° C. for 20 minutes. Lastly, the hydrogel fibers were rehydrated in 1× Tris-buffered saline (TBS) buffer (Sigma-Aldrich T1503, 99.9% and Sigma-Aldrich T3253, 99.0%) for further use. The compositions of all fabricated fibers are provided in Table. 2.

TABLE 2

| | TEOS:HCl:H2O | TEOS wt. % in | HCL wt. % in | GA wt. % in | PVA wt. % in |
|---|---|---|---|---|---|
| Nomenclatura | (molar ratio) | fibers | fibers | fibers | fibers |
| 10P-1T-GA | 1:4:4 | 1 | 0.007 | 0.005 | 10 |
| 10P-2T-GA | 2:4:8 | 2 | 0.007 | 0.005 | 10 |
| 10P-3T-GA | 3:4:12 | 3 | 0.007 | 0.005 | 10 |
| 10P-1T-GA | 4:4:16 | 4 | 0.007 | 0.005 | 10 |

Core-clad optical probe fabrication. As-fabricated optical fiber probes were dried and re-inserted into a silicone tubing (Mcmaster carr, 51845K66) and reswelled in water. 100 μL GA was added into 10 g of 5 wt. % PVA pre-solution and mixed in the mixer at 2000 rpm for 1 minute with a vacuum level at 16 kPa. 150 μL GA was added into 10 g of 5 wt. % PVA pre-solution and mixed in the mixer at 2000 rpm for 1 minute with a vacuum level at 16 kPa. The above two solution were mixed (1:1) together and spun at 2000 rpm for 1 minute with a vacuum level at 16 kPa. The mixed solution was infused into the silicone tubing and allowed to cross-link for 4 hours at room temperature. Core-clad optical fiber probes were extruded by submerging in DCM and stored in water for further use.

XRD characterization of hydrogel materials. X-ray scattering measurement was performed by SAXSLAB GANESHA 300XL instrument with a Dectris Pilatus 300K 2D CMOS photon counting detector (size 83.8×106.5 mm²). A small angle 2 mm beamstop was used for SAXS measurements and a wide angle 2 mm beamstop was used for WAXS measurements. The exposure time was 600s. The average size of the nanocrystalline domain was calculated by the Scherrer's equation D=kλ/(β·cos θ) where k is a dimensionless shape factor varying with the actual shape of the nanocrystalline domain (k=1, approximating the spherical shape of the nanocrystalline domains), λ is the wavelength of X-ray diffraction (λ=1.54 Å), and θ is the peak of the Bragg angle, β is the full width at half maximum (FWHM) of the WAXS peaks. The d-spacing between nanocrystalline domains was calculated by d=2π/qₘₐₓ, where qₘₐₓ is the q value at its maximum intensity from SAXS patterns. The FWHM β and qₘₐₓ were obtained by curve fitting of the WAXS and SAXS patterns in Origin (OriginLab Corporation), respectively.

DSC characterization of hydrogel materials. The crystallinity of hydrogel fibers and materials was evaluated by a DSC instrument (2920 TA instrument). The PVA hydrogels were measured in the dry state. A small amount of sample (1-15 mg) was contained within a crucible (TA instrument T81006) and placed into a temperature-controlled DSC cell. A blank crucible was used as a reference. The sample was heated up from 30° C. to 300° C. in air with a heating rate of 20° C./min. The differential heat flow to the sample and reference was monitored and recorded by the instrument. To obtain the melting fusion enthalpy of endothermic peaks, heat flow (mW) over sample weight (mg) was plotted against time (s). The areas of melting endothermic peaks were integrated in Origin (OriginLab Corporation). The degree of crystallinity a can be estimated from the formula: α=100%·ΔHₘ/ΔH_{f}, where ΔHₘ (J/g) is calculated from the integration of melting endothermic peaks and ΔH_{f} (150 J/g) is the enthalpy of melting 100% of PVA crystallites. The crystallinity results of PVA samples are provided in Table 3.

TABLE 3

| | ΔHm (mJ/mg) | ΔH_{f} (mJ/mg) | Crystallinity % |
|---|---|---|---|
| PVA | 40.90 ± 20.56 | 150 | 28.4 ± 3.5 |
| COMPACT (−) | 32.39 ± 1.63 | 150 | 21.6 ± 1.1 |
| COMPACT (+) | 19.12 ± 2.21 | 150 | 12.7 ± 1.5 |

Hydrogel Refractive Index Measurement. A series of hydrogel membranes were prepared via spin coating (KW-4A Spin, SETCAS) on silicon substrates (UniversityWafer, Inc., Model 447). Si substrates were cut into square wafers (13.5 mm×17.5 mm) using a diamond cutter. Si wafers were washed and ultrasonicated in Acetone (Sigma-Aldrich 179124, 99.5%) for 3 minutes. They were rinsed with MilliQ water after removing excess Acetone followed by washing and ultrasonication in 30 wt. % H₂SO₄ solution for 3 minutes. They were rinsed by MilliQ water after removing excess H₂SO₄ solution followed by washing and ultrasonication in 10 wt. % of H₂O₂ solution (Sigma-Aldrich 216763, 30 wt. % in water) for 3 minutes. 95% of Ethanol (concentration and company) was used to rinse the Si wafers to make sure the surfaces are hydrophilic. Si wafers were loaded on the spin coater and 10P-GA, 10P-1T-GA, 10P-2T-GA, 10P-3T-GA, and 10P-4T-GA membranes (n=4 for each group) were prepared at 1000 rpm for 10 s, and at 5000 rpm 50s. PVA solutions used for the membranes were prepared by the same method as discussed before. PVA membrane-coated Si wafers were allowed to cross-link and dry in the air for at least 12 hours and annealed at 100° C. for 20 minutes. PVA membrane-coated Si wafers were loaded on an ellipsometer to obtain the refractive index in the range of 400 nm to 700 nm. Alignment and calibration of the reflection beam were conducted for each wafer before each measurement. The measurements were carried out on the membranes in their dry states.

Hydrogel Absorbance and Fluorescence Measurement. A series of hydrogel membranes (denoted as 10P-GA, 10P-1T-GA, 1OP-2T-GA, 1OP-3T-GA, 1OP-4T-GA, n=4 for each group) were prepared and cross-linked in a 96-well plate via similar synthesis methods as discussed above. 1 mL of PVA solutions were added to each well and allowed to cross-link and dry in the air for at least 12 hours followed by annealing at 100° C. for 20 minutes. 100 μL of MilliQ water was added to each well to rehydrate the membranes. We used 200 μL of MilliQ water in blank wells as the blank control. The 96-well plate was placed in a plate reader (Biotek Synergy 2) to obtain transmittance spectra in the range of 400 nm to 700 nm. The autofluorescence readings were acquired at 470 nm excitation/510 nm emission and 485 nm excitation/520 nm emission, respectively. PVA membranes were measured in thickness 3 times by a caliper to normalize the transmittance spectra and autofluorescence readings regarding thickness.

All the fibers were maintained hydration prior to the extension test. A tensile instrument (Stable Micro System, TA. XT plusC, 50N load cell) was used. The hydrogel fibers were stretched by tensile tests at a tensile rate of 1 mm/second. The nominal stress was calculated from the formula σ=F/A, where F is the force recorded by the instrument and A is the cross-sectional area of the fibers in the hydrated state. The strain was calculated through ϵ=ΔL/L, where ΔL is the displacement and L is the gauge length. Two distinct marks were labeled with on the fibers and the initial gauge length L was determined as the distance between two marks prior to the tensile test. A high-resolution camera was used to record the entire tensile process for displacement tracking. The stress-strain curve was plotted based on calculated nominal stress and strain. The elastic moduli (E) were determined by calculating the average slope of the stress-strain relationship in the first 10% of applied strain. The average slope is determined by linear regression (OriginLab Corporation). The stretchability (%) of fibers were reported at the strain from stress-strain curves where the fibers snapped (fracture point).

Light attenuation of hydrogel fibers. The light transmission loss of hydrogel fibers was tested by the cutback method. The ferrule-connected hydrogel fibers were inserted into a plastic tube (5 cm in length, 3 mm in diameter) and injected with 1 wt. % agar gel to maintain the hydrated state of hydrogel fibers. The ferrule was connected to a 470 nm LED light (Thorlabs M470F3) through an adaptor (Thorlabs SM1FCM). The power (dB) of transmitted light through the hydrogel fiber was measured using a power meter (Thorlabs, PM16-122). The original power reading was recorded, and a 0.5 cm interval of cutting was adapted. Starting from the far end from the ferrule, the output power was measured after each cut by a cutter. The attenuation coefficient α was calculated by the formula

$$α = ( \frac{1 {0}^{4}}{{L}_{1} - {L}_{2}} ) \cdot log ( \frac{{P}_{1}}{{P}_{2}} )$$

where L₁ and L₂ are the original and cut lengths of the fiber in meters, respectively. P₁ and P₂ are the transmitted power reading before and after the cut, respectively.

Dimension measurements of hydrogel fibers. Microscopic images of hydrogels were acquired with a microscope (AmScope) in water under the bright field mode. Each fiber was taken images in three different locations (Two ends and the middle part). The diameter of the fibers was measured by Image J (9 measurements of each fiber) afterwards. The length of fibers was measured by a caliper (3 measurements of each fiber).

SEM imaging. The SEM images were acquired with dried samples by a scanning electron microscope (FEI Magellan 400 XHR). The as-fabricated integrated optrode probe was cut into thin pillars (0.1 mm in height) and mounted on carbon tape to examine the cross section of the probe.

Stability tests of hydrogel fibers. The fabricated 1OP-3T-GA fibers were incubated at 37° C. under physiological solutions (saline, ionic strength 305˜310 mOsm, pH from 6.0 to 8.0) over 12 weeks to validate the stability of hydrogel materials. The dimensions of fiber were measured before and after the incubation and statistical analysis was performed on the dimensions between pre-incubation and post-incubation each week.

Cell culture and biocompatibility tests. The HEK 293FT cell line was maintained in DMEM (with GlutaMax)+10% fetal bovine serum and seeded in a 24 well plate. COMPACT hydrogel fibers were incubated in DMEM for 24 hours at 37° C. Hydrogel incubated DMEM was then added into the well plate and incubated for 24 hours. Calcein-AM (green, Sigma-Aldrich 17783) was added to indicate living cells, and ethidium homodimer-1 (red, Sigma-Aldrich 46043) was added to indicate dead cells. A fluorescent microscope (Nikon TiU with SOLA Light Engine Gen III illumination hardware and PCO panda sCMOS camera) was used to take images of cells with and without hydrogel incubation.

Animals. All animal surgeries were reviewed and approved by the Committee on Animal Care at the University of Massachusetts Amherst. Wild-type mice (C57BL/6J) were purchased from the Jackson Laboratory. Mice were given ad libitum access to food and water and were housed at 24° C.±1° C., with 50% relative humidity, and on a 12-h light/12-h dark cycle.

In vivo hydrogel fiber implantation into the mouse brain. Mice (C57BL/6J) were anesthetized in a chamber with isoflurane and then positioned in a stereotactic frame (RWD Life Science) with a heating pad to maintain the body temperature of mice. All the surgeries were carried out under aseptic conditions and isoflurane was provided to the mice to maintain the anesthetized state. Eye cream was applied to maintain the moisture of the eyes. The hair on the head was removed by hair removing cream and a skin incision was made to expose the skull. Lambda and bregma points were used to align the skull with respect to the mouse brain atlas [Allen Brain Atlas]. Viral injection and fiber implantation coordinates (Nucleus accumbens (NAc), anteroposterior (AP): +1.25 mm, mediolateral (ML): ±0.8 mm, dorsoventral (DV): −4.5 mm) were established according to the mouse brain atlas. An opening was made by a micro drill (RWD Life Science) on the skull according to the coordinates. 600 nL of adeno-associated virus (AAV) carrying hSyn::GCaMP6s was injected through a micro syringe and a micro pump (World Precision Instruments, Micro 4). The viral injection device was lifted by 0.2 mm and maintained in the VTA region for 15 minutes to permit the diffusion of the virus to the target region. At the end of fiber probe insertion, the fiber probes were lifted by 0.1 mm to accommodate the virus volume. Last, fiber probes were fixed on the skull by the adhesive (Parkell, C&B METABOND) and reinforced by dental cement (Jet Set-4). The mice were monitored on the heating pad after isoflurane was removed until fully awakened.

Thy1:ChR2-EYFP mice were anesthetized in a chamber with isoflurane and then positioned in a stereotactic frame (RWD Life Science) with a heating pad to maintain the body temperature of mice. stereotactic frame (RWD Life Science) with a heating pad to maintain the body temperature of mice. All the surgeries were carried out under aseptic conditions and isoflurane was provided to the mice to maintain the anesthetized state. Allen brain atlas was used to align the skull and find the coordinates. Optrode device coordinates (VTA, AP: −3.00 nm, ML: +(or −) 0.45 mm, DV: −4.80 mm) were established based on the mouse brain atlas. Before the optrode implantation, an opening was made (AP: −3.5 mm, ML: − (or +) 1.5 mm, DV: −0.2 mm) for ground screw implantation and cerebrospinal fluid was contacted with the screw. In the end, optrode devices were fixed on the skull by the adhesive (Parkell, C&B METABOND) and reinforced by dental cement (Jet Set-4). The mice were monitored on the heating pad after isoflurane was removed until fully awakened.

After 4 weeks of recovery, mice were tethered with the fiber photometry (FIP) system. A ferrule (Thorlabs CF440) was fixed with the silica fiber on the other end. A connecting sleeve (Thorlabs ADAF1) was used to couple the silica fiber on the ferrule end and the implanted fiber probe. The mice were placed in a custom-made chamber (20×20×20 cm) for social preference tests. Custom-written python codes were used to compute fluorescent signals. A 470 nm LED (Thorlabs M470F3) and a 405 nm LED (Thorlabs M405F3) were coupled to a custom setup with dichroic mirrors (Thorlabs DMLP425R) on an optical breadboard. To extract 405 and 470 nm signals, illumination periods were determined by detecting synchronization ON/OFF pulses for each LED. Each illumination contains pulses at 10 Hz. To eliminate the moving artifacts, the fitted 470 nm signals were subtracted from the fitted 405 nm signals.

In all the behavioral experiments, adult mice implanted with optical fiber probes (C57BL/6, 4 weeks after implantation) were acclimated to the behavior chamber for >30 minutes before testing. Adult mice (C57BL/6, aged 5-6 weeks) with the same sex were used as strangers. A chamber box (20×20×20 cm) containing a social cage was used for the social interactions. Next, one novel mouse was introduced to the social zone. The test mouse was then exposed to the novel mouse and allowed to freely interact. GCaMP fluorescence change was recorded concurrently with the social tests. A dark-vision camera was set up above the social chamber for video footage recording during social tests. The amount of time spent interacting and the distance of social interaction were analyzed with customized algorithms for social interaction assessment with DeepLabCut. The analyzed social interaction epochs were correlated with GCaMP signals.

Immunohistology. The mice were deeply anesthetized through fatal plus (Vortech Pharmaceuticals, LTD) and transcardic perfusion was performed with 20 ml PBS (Sigma-Aldrich P3813) followed by 20 ml 4% paraformaldehyde (PFA, Sigma-Aldrich 8187151000) solutions. The brain tissues were then dissected from the bodies and fixed in 4% PFA solution at 4° C. overnight. The fixed tissues were then treated with 30% sucrose in PBS for 2 days. The brain tissues were quickly frozen at −20° C. in an O.C.T cube (21.5×21.5×22 mm) and sectioned on a cryostat (Leica CM1900) with a thickness of 20 μm. Slices were permeabilized in PBST (0.3% Triton-X-100 in PBS, Sigma-Aldrich 93443) for 15 minutes at room temperature. 200 μL of primary antibody solutions (Iba1 Rabbit, and GFAP Rabbit, Agilent Dako, Z0334, at a dilution of 1:400 in PBST) were used to stain and incubate slices overnight at room temperature. Slices were washed with PBST 3 times. Secondary antibody solutions (GFAP: Thermo Fisher Scientific, Donkey anti-Rabbit IgG (H+L) Highly Cross-Absorbed Secondary Antibody Alexa Fluor 488 Invitrogen, #A-21206. Iba1: Thermo Fisher Scientific, Donkey anti-Rabbit IgG (H+L) Highly Cross-Absorbed Secondary Antibody Alexa Fluor 555, #A-31572. Dilution: 1:200 in PBST) were used to stain slices at room temperature for 2 hours. Slices were washed by PBST 3 times and mounted on glass slides. DAPI mounting medium (Southernbiotech, Fluoromount-G, Cat. No. 0100-01) was applied on a coverglass and placed on top of a glass slide with the slices. Slides were left dried in the air at room temperature overnight. Images were taken from a confocal microscope (Leica SP2).

Electromyography. EMG signals were recorded from the gastrocnemius muscle with one reference needle electrode, one hydrogel working electrode (287±14 μm) and one ground electrode. A 473 nm laser (200 mW/mm², 0.5 Hz, pulse width 50 ms) was used for transdermal optical stimulation. EMG data triggered by optogenetic activation were collected through a DAM50 system.

In vivo electrophysiology. For electrophysiological recording, the pin connectors of optrode devices were connected to a recording system (DAM50). A 473 nm laser was connected to implanted optrode devices through a ferrule-sleeve-ferrule connecting system and used for optical illumination. During optical stimulation, a laser pulse with a 50 ms pulse width and a frequency of 0.5 Hz was used. Signals were recorded with 50 kHz sampling frequency and filtered in the range of 1-1000 Hz. Amplitude and noise level of evoked potentials were calculated with a MATLAB algorithm.

This disclosure further encompasses the following aspects.

Aspect 1: A miniaturized hydrogel comprising a reaction product of a hydroxyl-containing polymer, a primary crosslinker, and a secondary crosslinker; and water; wherein each of the primary crosslinker and the secondary crosslinker are capable of reacting with the hydroxyl-containing polymer; and wherein the hydrogel is made by a method comprising: contacting the hydroxyl-containing polymer, the primary crosslinker, and the secondary crosslinker under conditions effective to provide a crosslinked hydrogel; acidifying the crosslinked hydrogel; drying the crosslinked hydrogel under tension; and rehydrating the dried hydrogel to provide the miniaturized hydrogel.

Aspect 2: The miniaturized hydrogel of aspect 1, wherein the thickness of the hydrogel decreases when dried under tension, the hydrogel does not swell by more than 10% in any direction when rehydrated, and the thickness of the hydrogel is reduced by at least 70% when rehydrated compared to the initial hydrogel.

Aspect 3: The miniaturized hydrogel of aspect 1 or 2, wherein the hydroxyl-containing polymer comprises polyvinyl alcohol, poly(hydroxypropyl methacrylate), or a copolymer thereof.

Aspect 4: The miniaturized hydrogel of any of aspects 1 to 3, wherein the primary crosslinker comprises silicon, preferably wherein the primary crosslinker comprises a tetra(C₁₋₆ alkyl)orthosilicate.

Aspect 5: The miniaturized hydrogel of any of aspects 1 to 4, wherein the primary crosslinker comprises tetraethyl orthosilicate.

Aspect 6: The miniaturized hydrogel of any of aspects 1 to 5, wherein the secondary crosslinker comprises a dialdehyde, preferably a (C₃₋₁₈ alkylene) dialdehyde, a dicarboxylic acid, preferably a (C₃₋₆ alkylene dicarboxylic acid), or a combination thereof.

Aspect 7: The miniaturized hydrogel of any of aspects 1 to 6, wherein the secondary crosslinker comprises a dialdehyde comprising glutaraldehyde.

Aspect 8: The miniaturized hydrogel of any of aspects 1 to 7, wherein the hydrogel comprises a plurality of nanocrystalline domains, wherein the nanocrystalline domains are present in an amount effective to provide the hydrogel with a total crystallinity of 5 to 20%.

Aspect 9: The miniaturized hydrogel of any of aspects 1 to 8, further comprising a conductive filler, preferably carbon nanotubes.

Aspect 10: The miniaturized hydrogel of any of aspects 1 to 9, wherein the hydrogel is in the form of a fiber, preferably having an average diameter of 50 to 500 micrometers, preferably 50 to 300 micrometers.

Aspect 11: The miniaturized hydrogel of aspect 10, further comprising an outer layer on the surface of the hydrogel fiber, preferably wherein the outer layer comprising a crosslinked hydroxyl-containing polymer and a filler.

Aspect 12: The miniaturized hydrogel of any of aspects 1 to 11, comprising 45 to 65, or 50 to 60, or 55 to 60 weight percent of the reaction product of the hydroxyl-containing polymer, the primary crosslinker, and the secondary crosslinker; and 35 to 55, or 40 to 50, or 40 to 45 weight percent water, wherein weight percent is based on the total weight of the miniaturized hydrogel.

Aspect 13: The miniaturized hydrogel of any of aspects 1 to 12, wherein the primary crosslinker is present in an amount of greater than 0 to 5 weight percent, preferably 1 to 4 weight percent, or 1 to 3 weight percent, each based on the total weight of the miniaturized hydrogel.

Aspect 14: The miniaturized hydrogel of any of aspects 1 to 13, wherein the miniaturized hydrogel exhibits: a stretchability of greater than 100%, preferably 100 to 300%, more preferably 100 to 250%; an elastic modulus of less than 35 MPa, or 20 to 32 MPa; or both.

Aspect 15: The miniaturized hydrogel of any of aspects 1 to 14, wherein the miniaturized hydrogel has a refractive index of 1.35 to 1.45 at 480 nanometers.

Aspect 16: The miniaturized hydrogel of any of aspects 1 to 15, wherein the miniaturized hydrogel exhibits a light transmission of greater than 95%.

Aspect 17: A miniaturized hydrogel comprising: 45 to 65, or 50 to 60, or 55 to 60 weight percent of a reaction product of a hydroxyl-containing polymer, a primary crosslinker, and a secondary crosslinker; and 35 to 55, or 40 to 50, or 40 to 45 weight percent water; wherein weight percent is based on the total weight of the miniaturized hydrogel; wherein the hydrogel comprises a plurality of nanocrystalline domains, wherein the nanocrystalline domains are present in an amount effective to provide the hydrogel with a total crystallinity of 5 to 20%; wherein the miniaturized hydrogel exhibits one or more of: a stretchability of greater than 100%, preferably 100 to 300%, more preferably 100 to 250%; or an elastic modulus of less than 35 MPa, or 20 to 32 MPa; or a refractive index of 1.35 to 1.45 at 480 nanometers; or a light transmission of greater than 95%.

Aspect 18: A neural probe comprising the miniaturized hydrogel of any of aspects 1 to 17.

Aspect 19: A microelectrode comprising the miniaturized hydrogel of any of aspects 1 to 17.

Aspect 20: An implantable medical device comprising the miniaturized hydrogel of any of aspects 1 to 17.

Aspect 21: A method for the manufacture of the miniaturized hydrogel of any of aspects 1 to 17, the method comprising: contacting a hydroxyl-containing polymer, an inorganic crosslinker, and a second crosslinker under conditions effective to provide a crosslinked hydrogel; acidifying the crosslinked hydrogel; drying the crosslinked hydrogel under tension; and rehydrating the dried hydrogel to provide the miniaturized hydrogel.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other. “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “an aspect” means that a particular element described in connection with the aspect is included in at least one aspect described herein, and may or may not be present in other aspects. The term “combination thereof” as used herein includes one or more of the listed elements, and is open, allowing the presence of one or more like elements not named. In addition, it is to be understood that the described elements may be combined in any suitable manner in the various aspects.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A miniaturized hydrogel comprising
a reaction product of
a hydroxyl-containing polymer,
a primary crosslinker, and
a secondary crosslinker; and
water;
wherein each of the primary crosslinker and the secondary crosslinker are reactive towards the hydroxyl-containing polymer; and
wherein the hydrogel is made by a method comprising:
contacting the hydroxyl-containing polymer, the primary crosslinker, and the secondary crosslinker under conditions effective to provide a crosslinked hydrogel;
acidifying the crosslinked hydrogel;
drying the crosslinked hydrogel under tension; and
rehydrating the dried hydrogel to provide the miniaturized hydrogel.

2. The miniaturized hydrogel of claim 1, wherein the thickness of the hydrogel decreases when dried under tension, the hydrogel does not swell by more than 10% in any direction when rehydrated, and the thickness of the hydrogel is reduced by at least 70% when rehydrated compared to the initial hydrogel.

3. The miniaturized hydrogel of claim 1, wherein the hydroxyl-containing polymer comprises polyvinyl alcohol, poly(hydroxypropyl methacrylate), or a copolymer thereof.

4. The miniaturized hydrogel of claim 1, wherein the primary crosslinker comprises silicon.

5. The miniaturized hydrogel of claim 4, wherein the primary crosslinker comprises a tetra(C₁₋₆ alkyl)orthosilicate.

6. The miniaturized hydrogel of claim 1, wherein the secondary crosslinker comprises a dialdehyde.

7. The miniaturized hydrogel of claim 1, wherein the secondary crosslinker comprises a (C₃₋₁₈ alkylene) dialdehyde, a dicarboxylic acid, or a combination thereof.

8. The miniaturized hydrogel of claim 1, wherein the hydrogel comprises a plurality of nanocrystalline domains, wherein the nanocrystalline domains are present in an amount effective to provide the hydrogel with a total crystallinity of 5 to 20%.

9. The miniaturized hydrogel of claim 1, further comprising a conductive filler.

10. The miniaturized hydrogel of claim 1, wherein the hydrogel is in the form of a fiber having an average diameter of 50 to 500 micrometers.

11. The miniaturized hydrogel of claim 10, further comprising an outer layer on the surface of the hydrogel fiber, wherein the outer layer comprises a crosslinked hydroxyl-containing polymer and a filler.

12. The miniaturized hydrogel of claim 1, comprising
45 to 65 weight percent of the reaction product of the hydroxyl-containing polymer, the primary crosslinker, and the secondary crosslinker; and
35 to 55 weight percent water,
wherein weight percent is based on the total weight of the miniaturized hydrogel.

13. The miniaturized hydrogel of claim 1, wherein the primary crosslinker is present in an amount of greater than 0 to 5 weight percent, based on the total weight of the miniaturized hydrogel.

14. The miniaturized hydrogel of claim 1, wherein the miniaturized hydrogel exhibits:
a stretchability of greater than 100%; or
an elastic modulus of less than 35 MPa;
or both.

15. The miniaturized hydrogel of claim 1, wherein the miniaturized hydrogel has a refractive index of 1.35 to 1.45 at 480 nanometers.

16. The miniaturized hydrogel of claim 1, wherein the miniaturized hydrogel exhibits a light transmission of greater than 95%.

17. A miniaturized hydrogel comprising:
45 to 65 weight percent of a reaction product of a hydroxyl-containing polymer, a primary crosslinker, and a secondary crosslinker; and
35 to 55 weight percent water;
wherein weight percent is based on the total weight of the miniaturized hydrogel;
wherein the hydrogel comprises a plurality of nanocrystalline domains, wherein the nanocrystalline domains are present in an amount effective to provide the hydrogel with a total crystallinity of 5 to 20%;
wherein the miniaturized hydrogel exhibits one or more of:
a stretchability of greater than 100%;
an elastic modulus of less than 35 MPa;
a refractive index of 1.35 to 1.45 at 480 nanometers; and
a light transmission of greater than 95%.

18. A neural probe or a microelectrode comprising the miniaturized hydrogel of claim 1.

19. An implantable medical device comprising the miniaturized hydrogel of claim 1.

20. A method for the manufacture of the miniaturized hydrogel of claim 1, the method comprising:
contacting a hydroxyl-containing polymer, a primary crosslinker, and a secondary crosslinker under conditions effective to provide a crosslinked hydrogel;
acidifying the crosslinked hydrogel;
drying the crosslinked hydrogel under tension; and
rehydrating the dried hydrogel to provide the miniaturized hydrogel.
